(19) United States
(12) Patent Application Publication
(10) Pub. No.: US 2012/0244567 A1
(43) Pub. Date: Sep. 27, 2012

(54) HUMAN EMBRYONIC STEM CELLS FOR HIGH THROUGHOUT DRUG SCREENING

(75) Inventor: Xianmin Zeng, Novato, CA (US)

(73) Assignee: BUCK INSTITUTE FOR RESEARCH ON AGING, Novato, CA (US)

(21) Appl. No.: 13/392,487

(22) PCT Filed: Sep. 3, 2010
(86) PCT No.: PCT/US10/47893

§ 371 Date: Jun. 13, 2012

Related U.S. Application Data
(60) Provisional application No. 61/240,097, filed on Sep. 4, 2009.

(51) Int. Cl. C12N 5/0735; C12N 5/071; C12Q 1/32; C12Q 1/48; C12N 5/0797; C12Q 1/18

(52) U.S. Cl. 435/15; 435/366; 435/405; 435/32; 435/26; 435/325; 435/377

(57) ABSTRACT

Methods of culturing embryonic stem cells in a format suitable for high-throughput screening (HTS) are provided. In addition compounds that show differential cytotoxic/protective activity on embryonic stem cells (ESCs) and neurological stem cells (NSCs) are provided.

### CROSS-REFERENCE TO RELATED APPLICATIONS

This application claims benefit of and priority to U.S. Ser. No. 61/240,097, filed on Sep. 4, 2009, which is incorporated herein by reference in its entirety for all purposes.

### STATEMENT OF GOVERNMENTAL SUPPORT

[Not Applicable]

### FIELD OF THE INVENTION

The present invention relates to the fields of cell biology and neurobiology. Methods of culturing embryonic stem cells in a format suitable for high-throughput screening (HTS) are provided.

### BACKGROUND OF THE INVENTION

The ability to expand human embryonic stem cells (hESCs) unlimitedly in culture and to differentiate them into specific somatic cell types (Thomson et al. (1998) Science. 282: 1145-1147) make them a useful tool in the development of hESC-based automated screening platforms for drug discovery. Although this possibility has not yet attracted as much attention as the ideas of cell replacement, personalized medicine and other more direct clinical applications, hESCs are superior to most commonly used cell-culture models of drug discovery which employ tumor-derived or immortalized cell lines or primary cell culture. This is because tumor-derived and immortalized cells are often karyotypically abnormal and may diverge physiologically from normal cells in various respects, whereas primary cells have limited capacity for expansion.

Culturing hESCs and their differentiated neural derivatives in defined media in a format amendable for HTS been demonstrated to be technically difficult and, to our knowledge, there has no report on hESC-based HTS in the literature.

### SUMMARY OF THE INVENTION

In certain embodiments methods are provided for feeder-free culture of pluripotent stem cells (e.g., hESCs, iPSCs, etc.) and hESC-derived and/or iPSC-derived neural stem cells (NSCs) in formats suitable for high throughput screening (HTS). The methods readily permit measurement of standard HTS endpoints using, for example, ATP and/or LDH assays that are indicative of differentiation processes or toxicity.

In addition, it was discovered that compound exist that show differential toxicity in pluripotent stem cells (e.g., hESCs, iPSCs) and multipotent stem cells (e.g., hESC-derived NSCs). In particular compounds are identified that can specifically or preferentially kill either hESCs or NSC or both. Compounds exhibiting differentially toxicity to these cells types have numerous applications including, but not limited to preparation of pure cell populations.

In certain embodiments methods are provided for culturing human embryonic stem cells (hESCs) in a feeder-free format compatible with high throughput screening. The methods typically involve providing human embryonic stem cells in a vessel coated with extracellular matrix material (e.g., MATRIGEL™); and culturing said stem cells in medium comprising Dulbecco's Modified Eagle's medium/Ham's F12 supplemented with one or more of the following: knockout serum replacement, non-essential amino acids; L-glutamine, β-mercaptoethanol, an antibiotic; and basic fibroblast growth factor; where the medium is conditioned with embryonic fibroblasts. In certain embodiments the embryonic fibroblasts are mouse embryonic fibroblasts. In certain embodiments the medium is conditioned for at least 2 hours, at least 4 hours, at least 6 hours, at least 8 hours, at least 10 hours, at least 12 hours, at least 18 hours, or at least 24 hours. In certain embodiments the pluripotent cell is an embryonic stem cell (ESC), a human embryonic stem cell (hESC), an induced pluripotent stem cell iPSC, or a human induced pluripotent stem cell iPSC. In certain embodiments the medium is condition with mouse embryonic fibroblasts. In certain embodiments the knockout serum replacement comprises from about 5% to about 20% of the culture medium. In certain embodiments the knockout serum replacement comprises about 20% of the culture medium. In certain embodiments the non-essential amino acids range from about 1 mM to about 2 mM in the culture medium. In certain embodiments the non-essential amino acids are about 2 mM in the culture medium. In certain embodiments the L-glutamine ranges from about 1 mM to about 8 mM in the culture medium. In certain embodiments the L-glutamine comprises about 4 mM in the culture medium. In certain embodiments the β-mercaptoethanol ranges from about 0.01, 0.05, or about 0.1 mM to about 1 mM in the culture medium. In certain embodiments the β-mercaptoethanol comprises about 0.1 mM in the culture medium. In certain embodiments the antibiotic ranges from about 50 μg/mL to about 100 μg/mL in the culture medium. In certain embodiments the antibiotic and comprises about 50 μg/mL in the culture medium. In certain embodiments the antibiotic comprises Penn-Strep. In certain embodiments the basic fibroblast growth factor ranges from about 1 ng/mL to about 30 ng/mL, or from about 4 ng/mL to about 20 ng/mL in the culture medium, or about 4 ng/mL in the culture medium. In certain embodiments the Dulbecco's Modified Eagle's medium/Ham's F12 medium is supplemented with about 20% knockout serum replacement; about 2 mM non-essential amino acids; about 4 mM L-glutamine; about 0.01 mM (3-mercaptoethanol; about 50 μg/mL Penn-Strep; and about 4 ng/mL basic fibroblast growth factor.

Methods are also provided of culturing neural stem cells (NSCs) in a feeder-free format compatible with high throughput screening. The methods typically involve providing neural stem cells in a vessel, well, or dish coated with an extracellular matrix glycoprotein (e.g., fibronectin); and culturing the stem cells in medium comprising DMEF/12 supplemented with N2 medium; non-essential amino acids; bFGF; and EGF. In certain embodiments the medium is supplemented with N2 ranging from about 0.5× to about 2×, 1.5×, or about 1×. In certain embodiments the medium is supplemented with 1×N2 medium. In certain embodiments the non-essential amino acids range from about 0.5 mM to about 4 mM, or about 1 mM to about 2 mM in the culture medium. In certain embodiments the non-essential amino acids are about 2 mM in the culture medium. In certain embodiments the bFGF ranges from about 5 ng/mL to about 100 ng/mL, or about 10 ng/mL to about 50 ng/mL in the culture medium. In certain embodiments the bFGF comprises about 20 ng/mL in the culture medium. In certain embodiments the EGF ranges from about 5 ng/mL to about 40 ng/mL, or about 10 ng/mL to about 20 ng/mL in the culture medium. In certain embodiments the EGF comprises about 20 ng/mL in the culture medium. In certain embodiments the medium is supplemented with about 1×N2 medium; about 2 mM non-essential amino acids; about 20 ng/mL of bFGF; and about 2 ng/mL of EGF.

Also provided are methods of screening an agent for the ability to selectively inhibit the growth and/or proliferation of pluripotent stem cells (e.g., hESCs, IPSCs, etc.) and/or neural stem cells. The method typically involves contacting the pluripotent stem cell with the test agent; contacting a multipotent and/or a terminally differentiated cell with the test agent; determining the cytotoxicity of the test agent on the pluripotent cell and on the multipotent and/or terminally differentiated cell; and selecting agents that are preferentially cytotoxic or protective to pluripotent cells over multipotent cells and/or selecting agents that are preferentially cytotoxic or protective to pluripotent cells and/or multipotent cells over terminally differentiated cells. In various embodiments the pluripotent cell is an embryonic stem cell (ESC), a human embryonic stem cell (hESC), an induced pluripotent stem cell iPSC, a human induced pluripotent stem cell iPSC, and the like. In certain embodiments multipotent cell is a progenitor cell or a neural stem cell. In certain embodiments the selecting comprises recording the identity of agents that are preferentially cytotoxic to ESCs over NSCs and/or preferentially cytotoxic to ESC and/or NSCs over terminally differentiated cells in a database of agents that to selectively inhibit the growth and/or proliferation of human embryonic stem cells and/or neural stem cells. In certain embodiments the selecting comprises storing to a computer readable medium the identity of agents that are preferentially cytotoxic or protective to ESCs over NSCs and/or preferentially cytotoxic or protective to ESC and/or NSCs over terminally differentiated cells in a database of agents that selectively inhibit the growth and/or proliferation of human embryonic stem cells and/or neural stem cells. In certain embodiments the computer readable medium is selected from the group consisting of a flash memory, a memristor memory, a magnetic storage medium, and an optical storage medium. In certain embodiments the selecting comprises listing to a computer monitor or to a printout the identity of agents that are preferentially cytotoxic or protective to ESCs over NSCs and/or preferentially cytotoxic or protective to ESC or iPSCs and/or NSCs over terminally differentiated cells in a database of agents that to selectively inhibit the growth and/or proliferation of human embryonic stem cells and/or neural stem cells. In certain embodiments the selecting comprises further screening the selected agents for cytotoxic activity on cell lines. In certain embodiments the method comprises contacting a neural stem cell (NSC) with the test agent. In certain embodiments the method comprises contacting a terminally differentiated cell with the test agent. In certain embodiments the terminally differentiated cell is a cell selected from the group consisting of a neuron, an astrocyte, and an oligodendrocyte. In various embodiments the determining the cytotoxicity comprises performing one or more assays selected from the group consisting of an ATP assay, a lactate dehydrogenase (LDH) assay, an adenylate kinase (AK) assay, a glucose 6-phosphate dehydrogenase (G6PD) assay, MTT assay, and an MTS assay. In certain embodiments the selecting comprises identifying the agent as an NSC killer if it shows cytotoxicity against NSCs with at least 1.5 fold or greater potency for NSCs than ESCs or iPSCs and shows at least a 25% reduction in viability of NSCs as compared to a control. In certain embodiments the selecting comprises identifying the agent as an NSC killer if it shows cytotoxicity against NSCs with at least 2-fold or 3-fold, or 5-fold or greater potency for NSCs than ESCs or iPSCs and shows at least a 25% reduction in viability of NSCs as compared to a control. In certain embodiments the selecting comprises identifying the agent as an NSC killer if it reduces ATP concentrations with at least 2-fold or more potency for NSCs than ESCs, and that NSC values are 50% or more below a control mean. In certain embodiments the selecting comprises identifying the agent as an ESC killer if there is any significant selectivity for affecting ATP levels in ESCs over NSCs. In certain embodiments the contacting an embryonic stem cell comprises culturing the embryonic stem cell according to the methods described herein. In certain embodiments the contacting a neural stem cell comprises culturing the neural stem cell according to the methods described herein.

In various embodiments methods of generating a substantially homogenous population of embryonic stem cells (ESCs), are provided. The methods typically involve providing a population of embryonic stem cells and contacting the population with an agent that preferentially kills neural stem cells (NSCs), where the agent is provided in an amount to preferentially kill NSCs without substantially diminishing the population of embryonic stem cells. In certain embodiments the agent is selected from the group consisting of amethopterin (r,s), methiazole, trifluridine, bisacodyl, lasalocid sodium salt, pyrimethamine, chelidonine (+), cantharidin, tomatine, sanguinarine, clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin (a shown), eburnamonine, emetine hydrochloride, edoxudine, tamoxifen citrate, cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, and acriflavinium hydrochloride. In certain embodiments the agent is selected from the group consisting of amethopterin (r,s), methiazole, trifluridine, bisacodyl, lasalocid sodium salt, pyrimethamine, chelidonine (+), clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin (a shown), eburnamonine, emetine hydrochloride, edoxudine, tamoxifen citrate.

In various embodiments methods are provided for generating a substantially homogenous population of adult stem cells derived from human embryonic stem cells (hESCs) or induced pluripotent stem cells. The methods typically invovel differentiating adult stem cells from a population of human embryonic stem cells or induced pluripotent stem cells to form a population of adult stem cells; and contacting the population with an agent that preferentially inhibits the growth or proliferation of human embryonic stem cells or induced pluripotent stem cells remaining in the population, thereby producing a substantially homogenous population of adult stem cells. In certain embodiments the population of human embryonic stem cells or induced pluripotent stem cells is a population of human embryonic stem cells and the agent is an agent that preferentially inhibits the growth or proliferation of human embryonic stem cells. In certain embodiments the adult stem cells are neural stem cells (NSCs). In certain embodiments the agent is selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, gbr 12909 dihydrochloride, (−)-levobunolol hydrochloride, camptothecine (s,+), puromycin dihydrochloride, doxorubicin hydrochloride, and paclitaxel. In certain embodiments the agent is selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, gbr 12909 dihydrochloride, and (−)-levobunolol hydrochloride. In certain embodiments the population of differentiated cells comprises a population of postmitotic neuron cells.

Methods are also provided for generating a substantially homogenous differentiated population of cells derived from human embryonic stem cells (hESCs) or induced pluripotent stem cells. The methods typically involve differentiating cells from a population of human embryonic stem cells or induced pluripotent stem cells to form a population of differentiated cells; and contacting the population with one or more agents that preferentially inhibit the growth or proliferation of human embryonic stem cells and/or induced pluripotent stem cells, and/or adult stem cells in the population, thereby producing a substantially homogenous differentiated population of cells. In certain embodiments the differentiating comprises differentiating cells from a from a population of human embryonic stem cells. In certain embodiments the population of differentiated cells is a population of differentiated neural cells. In certain embodiments the differentiated cells are selected from the group consisting of neurons, astrocytes and oligodendrocytes. In certain embodiments the contacting comprises contacting the population with an agent that is toxic to both ESCs and NSCs and/or contacting the population with an agent that is toxic to ESCs and an agent that is toxic to NSCs. In certain embodiments the contacting comprises contacting the population with an agent that is toxic to both ESCs and NSCs and the agent is selected from the group consisting of cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, acriflavinium hydrochloride, cantharidin, tomatine, sanguinarine, camptothecine (s,+), puromycin dihydrochloride, doxorubicin hydrochloride, and paclitaxel. In certain embodiments the contacting comprises contacting the population with an agent that is toxic to ESCs where the agent is selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, GBR 12909 dihydrochloride, (−)-levobunolol hydrochloride; and an agent that is toxic to NSCs or to both NSCs and ESCs, where the agent toxic to NSCs is selected from the group consisting of clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin (a shown), eburnamonine, emetine hydrochloride, edoxudine, tamoxifen citrate, amethopterin (r,s), methiazole, trifluridine, bisacodyl, lasalocid sodium salt, pyrimethamine, and chelidonine (+), and the agent toxic to both NSCs and ESCs is selected from the group consisting of cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, acriflavinium hydrochloride, cantharidin, tomatine, sanguinarine, camptothecine (s,+), puromycin dihydrochloride, doxorubicin hydrochloride, and paclitaxel.

In certain embodiments the contacting comprises contacting the population with an agent that is toxic to NSCs where the agent is selected from the group consisting of clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin (a shown), eburnamonine, emetine hydrochloride, edoxudine, tamoxifen citrate, amethopterin (r,s), methiazole, trifluridine, bisacodyl, lasalocid sodium salt, and pyrimethamine, chelidonine (+); and an agent that is toxic to ESCs or to both NSCs and ESCs, where the agent toxic ESCs where the agent is selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, GBR 12909 dihydrochloride, and (−)-levobunolol hydrochloride, and the agent toxic to both NSCs and ESCs is selected from the group consisting of cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, acriflavinium hydrochloride, cantharidin, tomatine, sanguinarine, camptothecine (s,+), puromycin dihydrochloride, doxorubicin hydrochloride, and paclitaxel. In certain embodiments the agent comprises an agent selected from the group consisting of selamectin, amiodarone HCL, and minocycline HCL, and an analogue thereof.

### DEFINITIONS

The term “embryonic stem cell” or “ESC” refers to stem cells derived from the inner cell mass of an early stage embryo known as a blastocyst. Human embryos reach the blastocyst stage 4-5 days post fertilization, at which time they consist of 50-150 cells. Embryonic Stem cells (ESCs) are pluripotent and able to differentiate into all derivatives of the three primary germ layers: ectoderm, endoderm, and mesoderm.

The term “adult stem cells” refers to undifferentiated cells, found throughout the body after embryonic development, that multiply by cell division to replenish dying cells and regenerate damaged tissues. Also known as somatic stem cells, they can be found in juvenile as well as adult animals and humans. Adult stem cells have the ability to divide or self-renew indefinitely, and generate all the cell types of the organ from which they originate, potentially regenerating the entire organ from a few cells.

The term “neural stem cell” or “NSC” refers to undifferentiated cells typically originating from the neuroectoderm that have the capacity both to perpetually self-renew without differentiating and to generate multiple types of lineage-restricted progenitors (LRP). LRPs can themselves undergo limited self-renewal, then ultimately differentiate into highly specialized cells that compose the nervous system. In certain embodiments the use of a wide variety of neuroepithelial or neurosphere preparations as a source of putative NSCs is also contemplated.

The term “induced pluripotent stem cell” (Baker (2007). Nature Reports Stem Cells. doi:10.1038/stemcells.2007.124), commonly abbreviated as iPS cells or iPSCs, are a type of pluripotent stem cell artificially derived from a non-pluripotent cell, typically an adult somatic cell, by inducing a “forced” expression of certain genes. Induced Pluripotent Stem Cells are believed to be identical to natural pluripotent stem cells, such as embryonic stem (ES) cells in many respects, such as the expression of certain stem cell genes and proteins, chromatin methylation patterns, doubling time, embryoid body formation, teratoma formation, viable chimera formation, and potency and differentiability. Methods of making iPSCs are well known to those of skill in the art (see, e.g., Yamanaka et al. (1002&) Nature, 448: 313-317; Zhou et al. (2009) Cell Stem Cell, 4(5): 381-384, and references therein).

“Pluripotent stem cells” include both ESCs and iPSCs. Pluripotency is the ability of the human embryonic stem cell to differentiate or become essentially any cell in the body. In contrast to pluripotent stemcells, many progenitor cells are multipotent, i.e. they are capable of differentiating into a limited number of tissue types.

### BRIEF DESCRIPTION OF THE DRAWINGS

FIG. 1, panels A-H show the morphology and expression of stem cell markers in hESCs and hESC-derived NSCs cultured in 96-well plates. Panels A-B: Typical undifferentiated hESC morphology 24 hours after plating (panel A) and 3 days after passaging (panel B). Panels C-D: Expression of pluripotent markers Oct4 in cells cultured in 96-well plates (panel C, Oct4=green, nuclei=blue) and colonies cultured in 60 mm dishes (panel D, Oct4=red, nuclei=blue). Panels E-F: Homogenous hESC-derived NSCs are morphologically similar whether cultured in 96 well plates (panel E) or larger 60 mm dishes (panel F). Panels G-H: Uniform expression of nestin in NSCs cultured in 96 well plates (panel G) and 60 mm dishes (panel H) Nestin=red, nuclei=blue.

FIG. 2, panels A-B, illustrate primary screens and retests with the NINDS collection. Panel A: ATP levels in hESCs and NSCs. Panel B: Dose response of NSC and hESC to selectively screened NINDS compounds. Hits obtained in the primary screen were retested and validated to be toxic to NSCs in a dose-responsive manner.

FIGS. 3-5, illustrate the validation of a compound in larger numbers of cells. FIG. 3, panels A-D: Expression of β-III tubulin (panel A) and TH (panel B,) in NSCs that had been differentiated for 4 weeks (panel C=nuclei, panel D=merge). FIG. 4, panels E-G: NSCs after 2 (panel E), 4 (panel F) and 8 (panel G) hours exposure to amiodarone HCl (FIG. 4, panels H-J) dopaminergic neurons after 2 (panel H), 4 (panel I) and 8 (panel J) hours exposure to amiodarone HCl. FIG. 5: Changes in ATP levels in hESC, NSC, and dopaminergic neurons after exposure to three doses of amiodarone HCl for 48 h.

FIG. 6 illustrates the effect of amiodarone HCl on glia cells. Human Astrocytes (HA, top panel) after 1, 26 and 48 hours exposure to amiodarone HCl and untreated cells. NSCs, either after 1, 2, 3 and 4 hours exposure to amiodarone HCl or left untreated are shown on the bottom panel for comparison.

FIG. 7, panels A-D, show the effect of solTNFα on NSC survival. Three concentrations of solTNFα (0.1 nM, 1 nM and 10 nM) were added to freshly seeded NSC cultures. Cells were evaluated up to 24 hours for signs of cell death. No increase in cell death relative to untreated cultures was observed in the cultures treated with solTNFα. The images taken of the cells treated with the highest concentration of solTNFα, 10 nM, are representative of data obtained for all concentrations and are shown at (panel A) 1 hour, (panel B) 4 hours and (panel C) 24 hours post cytokine treatment. Panel D: Untreated cells are shown at 24 hours for comparison.

FIG. 8. Gene expression analysis.

FIG. 9, panels A-E, GSEA analysis.

### DETAILED DESCRIPTION

In various embodiments this invention pertains to the development of stem cell (e.g., hESC, IPSC, etc.)-based automated screening. To enable the development of stem cell (e.g., hESC)-based automated screening a number of limitations surrounding stem cell culture were overcome.

In particular, in various culture systems described herein, human pluripotent stem cells (including ESCs and/or iPSCs) and their differentiated derivatives are cultured without feeder layers in a format that is amendable to automated screening such as in 6-, 12-, 24-, 48-, and 96 well culture plates. Unlike mouse embryonic stem cells (mESCs) which can be efficiently expanded and differentiated from single cells, pluripotent stem cells (e.g., hESCs) are routinely passaged as small clumps of cells or differentiated via embryoid bodies formed from tens to hundreds of cells (Thomson et al. (1998) Science. 282: 1145-1147)).

Utilizing the defined media described herein along with the methods that result in increased cloning efficiency of pluripotent stem cells, it is possible to culture such cells in large numbers. The methods permit the generation of homogeneous and lineage-specific differentiated populations from hESCs and/or IPSCs while culturing them in large numbers for prolonged periods.

In addition, given our extensive experiences in neuronal differentiation of hESCs (Zeng et al. (2006) Neuropsychopharmacology. 31: 2708-2715; Zeng et al. (2004) Stem Cells., 22: 925-940; Freed et al. (2008) PLoS ONE 3:e1422.) and the potential application of hESC- and/or IPSC-derived neurons in cell replacement therapies for neurodegenerative diseases, we designed a set of experiments aimed at developing an hESC- and/or IPSC-based automated assay for screening small molecules that have differential toxicity to hESC- and/or IPSC-derived NSCs and their differentiated neural progenies. We reasoned that the development of this assay would help identify chemical compounds that are useful for eliminating proliferating cells in potential hESC- and/or IPSC-derived cell therapy products.

To this end, we chose to use the National Institute of Neurodegenerative Diseases and Stroke (NINDS) collection of FDA-approved drugs for assay optimization and pilot screening. The bioactivity of the compounds in this library and the ready availability of individual compounds identified as hits for follow-up studies made this library ideal for pilot screenings. Furthermore, these routinely used drugs have been highly optimized to hit specific targets and in nearly all cases the mechanisms of action are known.

By comparative screening on hESCs and hESC-derived homogenous NSCs using the NINDS collection, we were able to identify are identified herein that have differential toxicity to both cell populations. Hits obtained in the primary screen were then retested and a small subset was assayed for dose-responsiveness. One confirmed dose-responsive compound, amiodarone HCl, was further tested for toxicity in postmitotic neurons. We found amiodarone HCL to be toxic to NSCs but not to postmitotic neurons, indicating its potential use for depleting proliferating NSCs in hESC-derived cell populations for possible neural transplantation.

Some of the important applications of hESC- and/or IPSC-based high-throughput screening systems (HTS) are to screen drugs that may be useful for eliminating proliferating cells in hESC- and/or IPSC-derived cell therapeutic products, and to identify compounds/small molecules that have neuroprotective effects which may lead to small molecule therapy for neurodegenerative diseases.

As described herein, in various embodiments, methods are provided for feeder-free culture of hESCs and/or IPSC and/or hESC-derived and/or IPSC-derived neural stem cells (NSCs) in 96-well (or other) formats suitable for HTS. The assays permit measurement of standard HTS endpoints using, for example, ATP and LDH assays that are indicative of differentiation processes or toxicity.

In addition methods are described and illustrated for the comparative screening of thousands of compounds for toxicity in hESCs, IPSCs, iPSC-derived and hESC-derived NSCs. The screens exemplified herein have identified FDA-approved drugs that can specifically kill either hESCs or NSC or both. Compounds exhibiting differentially toxicity to these cells types have potential application in the preparation of pure cell populations, e.g., as described herein. In addition, the various compounds described herein can produce differential toxicity and/or protective effects in terminal differentiated neurons such as dopaminergic neurons (e.g., which might be useful for cell replacement therapy for Parkinson's disease).

### Screening Systems.

In various embodiments methods are provided for culturing pluripotent stem cells (e.g., hESCs, IPSCs, etc.) in a feeder-free format compatible with high throughput screening and/or culturing neural stem cells (NSCs) in a feeder-free format compatible with high throughput screening.

In certain embodiments the method of culturing pluripotent stem cells (hESCs, iPSCs, etc.) comprises providing human embryonic stem cells and/or induced pluripotent stem cell (e.g., human iPSCs) in a culture vessel (e.g., 6 well, 24 well, 96 well, etc. cell culture plates) having one or more surfaces coated with an appropriate substrate such as an extracellular matrix or substitute therefore (e.g., MATRIGEL®). In certain embodiments the well(s) comprise one or more surfaces coated with MATRIGEL®. In various embodiments the pluripotent stem cells are cultured in medium comprising Dulbecco's Modified Eagle's medium/Ham's F12 supplemented with a fetal bovine serum replacement (e.g., knockout serum replacement (KSR), Gibco BRL). In certain embodiments the medium additionally contains non-essential amino acids; and/or L-glutamine, and/or basic fibroblast growth factor (bFGF). In certain embodiments the medium is condition with mouse embryonic fibroblasts for at least 12, preferably at least 24 hours prior to use. In certain embodiments the medium additionally comprises an SH donor (e.g., β-mercaptoethanol), and/or an antibiotic (e.g., Penn Strep).

In various embodiments the knockout serum replacement comprises from about 1%, or from about 2%, or from about 3%, or from about 4%, or from about 5% to about 10%, or to about 12%, or to about 15%, or to about 18%, or to about 20%, or to about 25%, preferably from about 5% or about 10% or about 15% to about 20% of the culture medium. In certain embodiments the knockout serum replacement comprises about 20% of said culture medium.

In various embodiments the non-essential amino acids range from about 0.1 mM, or from about 0.5 mM, or from about 1 mM to about 2 mM or to about 2.5 mM, preferably from about 1 mM to about 2 mM in said culture medium. In certain embodiments the non-essential amino acids comprise about 2 mM in the culture medium.

In various embodiments the L-glutamine ranges from about 1 mM, or from about 2 mM, or from about 3 mM to about 4 mM, or to about 6 mM, or to about 7 mM, or to about 8 mM, preferably about 1 mM to about 4 mM, or about 1 mM to about 2 mM in the culture medium. In certain embodiments the L-glutamine comprises about 4 mM in the culture medium.

In various embodiments β-mercaptoethanol ranges from about 0.01 mM, or from about 0.05 mM, or from about 0.1 mM to about 1 mM, or to about 1.5 mM, or to about 2 mM, preferably from about 0.1 mM to about 1 mM in the culture medium. In certain embodiments the β-mercaptoethanol comprises about 0.1 mM in the culture medium.

In various embodiments an antibiotic is present in sufficient quantity to inhibit bacterial and/or fungal growth. In certain embodiments the antibiotic is Penn-Strep and comprises from about 5 μg/mL, or from about 10 μg/mL, or from about 20 μg/mL, or from about 30 μg/mL, or from about 40 μg/mL, or from about 50 μg/mL to about 500 μg/mL, or to about 400 μg/mL, or to about 300 μg/mL, or to about 200 μg/mL, or to about 100 μg/mL in the culture medium, more preferably from about 50 μg/mL to about 100 μg/mL in the culture medium. In certain embodiments the Penn-Strep comprises about 50 μg/mL in the culture medium.

In various embodiments the basic fibroblast growth factor ranges from about 1 ng/mL, or from about 2 ng/mL, or from about 3 ng/mL, or from about 4 ng/mL to about 100 ng/mL, or to about 50 ng/mL, or to about 30 ng/mL, or to about 20 ng/mL in the culture medium, preferably from about 4 ng/mL to about 20 ng/mL. In certain embodiments the fibroblast growth factor comprises about 4 ng/mL in the culture medium.

In certain embodiments the Dulbecco's Modified Eagle's medium/Ham's F12 medium is supplemented with: about 20% knockout serum replacement; about 2 mM non-essential amino acids; about 4 mM L-glutamine; about 0.01 mM β-mercaptoethanol; about 50 μg/mL Penn-Strep; and about 4 ng/mL basic fibroblast growth factor.

In various embodiments methods of culturing neural stem cells (NSCs) in a feeder-free format compatible with high throughput screening involve providing neural stem cells in a culture vessel (e.g., 6 well, 24 well, 96 well, etc. cell culture plates) having one or more surfaces coated with an appropriate substrate such as an extracellular matrix, e.g., MATRIGEL®, and/or Fibronectin. The cells are cultured in a medium comprising DMEF/12 supplemented with: N2 medium; non-essential amino acids; bFGF; and epidermal growth factor (EGF).

In various embodiments the N2 medium comprises about 0.1×, or about 0.3 X, or about 0.5× to about 2×, or to about 1.5×, or to about 1×, preferably from about 0.5× to about 1×. In certain embodiments the culture medium is supplemented with 1×N2 medium. In certain embodiments other substantially equivalent media (e.g., B27) can supplement or replace the N2 medium.

In various embodiments the non-essential amino acids range from about 0.1 mM, about 0.5 mM, or about 1 mM to about 2 mM or about 2.5 mM, preferably from about 1 mM to about 2 mM in said culture medium. In certain embodiments the non-essential amino acids comprise about 2 mM in the culture medium.

In various embodiments the basic fibroblast growth factor ranges from about 1 ng/mL, or about 5 ng/mL, or about 10 ng/mL to about 150 ng/mL, or to about 100 ng/mL, or to about 50 ng/mL in the culture medium, preferably from about 10 ng/mL to about 50 ng/mL in the culture medium. In certain embodiments the fibroblast growth factor comprises about 20 ng/mL in the culture medium.

In various embodiments the epidermal growth factor ranges from about 1 ng/mL, or about 5 ng/mL, or about 10 ng/mL to about 150 ng/mL, or to about 100 ng/mL, or to about 50 ng/mL in the culture medium, preferably from about 10 ng/mL to about 50 ng/mL, or to about 20 ng/mL in the culture medium, preferably from about 10 ng/mL to about 20 ng/mL in the culture medium. In certain embodiments the epidermal growth factor comprises about 20 ng/mL in the culture medium.

In certain embodiments the medium is supplemented with: about 1×N2 medium; about 2 mM non-essential amino acids; about 20 ng/mL of bFGF; and about 2 ng/mL of EGF.

Screening for Agents to Selectively Inhibit Growth and/or Proliferation of Human Embryonic Stem Cells and/or Neural Stem Cells.

It was a surprising discovery that certain compounds can show differential activity on pluripotent stem cells (e.g., ESCs, iPSCs, etc.) and progenitor cells (e.g., neural stem cells (NSCs)), and/or on terminally differentiated cells. Accordingly, methods are provided for screening for agents to selectively inhibit growth and/or proliferation of human embryonic stem cells and/or neural stem cells.

In certain embodiments the methods involve contacting a pluripotent stem cell (e.g., ESC, iPSC, etc.) with the test agent; contacting a progenitor cell (e.g., a neural stem cell (NSC)) and/or a terminally differentiated cell with the test agent; and determining the cytotoxicity of the test agent on the pluripotent stem cell (e.g., hESC) and on the progenitor (e.g., NSC) and/or terminally differentiated cell; and selecting agents that are preferentially cytotoxic to pluripotent stem cells (e.g., ESCs, iPSCs, etc.) over progenitors (e.g., NSCs) and/or selecting agents that are preferentially cytotoxic to pluripotent stem cells (e.g., ESCs, iPSCs) and/or NSCs over terminally differentiated cells. In various embodiments the cells (e.g., ESCs, iPSCs, NSCs, etc.) are cultured according to the culture methods described herein.

Cytotoxicity and/or metabolic activity can be measured by any of a number of convenient assays. For example, metabolic activity can be measured using an ATP assay to determine ATP content and/or activity in the subject cells. Other assays include, for example, the presence of intracellular enzymes such as lactate dehydrogenase (LDH), adenylate kinase (AK), glucose 6-phosphate dehydrogenase (G6PD), and the like in the culture supernatant. When cell membranes are compromised they become porous and allow these stable macromolecules to leak out and be quantitated using a variety of fluorescent, luminescent, and colorimetric assays. Similar assays pre-load cells with a radioactive (⁵¹Cr) or non-radioactive substance (usually an ester that is cleaved to a non-membrane-permeable product), and then measure the amount released into the supernatant upon loss of membrane integrity (such assays are often used in cell-mediated cytotoxicity assays). Other viability assays being used to measure cytotoxicity rely on the fact that adherent cells generally let go of their plastic substrate when they die. Dead cells are washed away, and the remaining cells are counted or otherwise quantitated.

Assays in common use for determining cytotoxicity fall into several categories. One category is “release” assays, in which a substance released by dying cells is measured. Often the substance is an enzyme, such as lactate dehydrogenase (LDH), adenylate kinase (AK), glucose 6-phosphate dehydrogenase (G6PD), and the like in the culture supernatant. When cell membranes are compromised they become porous and allow these stable macromolecules to leak out and be quantitated using a variety of fluorescent, luminescent, and colorimetric assays. Traditional enzyme-release assays have exploited the fact that these enzymes create NADH, which can be observed by UV spectroscopy at 340 nm. An alternative is to couple production of NADH to generation of a colored dye, as in the LDH-based CELLTITER® assays currently available from Promega. Other enzymes used in this way include, but are not limited to, phosphatases, transaminases, and argininosuccinate lyase.

Similar release assays involve pretreatment of the target cells with a radioactive isotope, generally ⁵¹Cr or ³H. Upon lysis, the radioactive contents are released and counted in a scintillation counter. The same process can also be carried out with fluorescent dyes, such as bis-carboxyethyl-carboxyfluorescein, calcein-AM, and the like.

Another type of release assay is the luminescent assay of ATP released from dead or damaged cells. This assay is often used as a proliferation assay, and it is discussed further below along with other proliferation assays.

Other viability assays being used to measure cytotoxicity rely on the fact that adherent cells generally let go of their plastic substrate when they die—dead cells are washed away, and the remaining cells are counted or otherwise quantitated.

Another category of cytotoxicity assay makes use of dyes that are able to invade dead cells, but not living cells. An example of such a dye is trypan blue.

Yet another category of cytotoxicity assays includes those methods directly related to apoptosis. These assays typically look for either protein markers of apoptotic processes or particular effects on DNA that are uniquely associated with apoptosis. Another method of studying apoptosis is to look at the ATP:ADP ratios in a cell, which change in a distinct way as the cell enters apoptosis. These assays may be performed by coupled luminescent methods (see, e.g., Bradbury et al. (2000) J. Immunol. Meth., 240: 79-92).

The MTT assay and the MTS assay are laboratory tests and standard colorimetric assays (an assay which measures changes in color) for measuring the activity of enzymes that reduce MTT or MTS+PMS to formazan, giving a purple color. It can also be used to determine cytotoxicity of potential medicinal agents and other toxic materials, since those agents would result in cell toxicity and therefore metabolic dysfunction and therefore decreased performance in the assay.

Yellow MTT (3-(4,5-Dimethylthiazol-2-yl)-2,5-diphenyltetrazolium bromide, a tetrazole) is reduced to purple formazan in living cells.[1] A solubilization solution (usually either dimethyl sulfoxide, an acidified ethanol solution, or a solution of the detergent sodium dodecyl sulfate in diluted hydrochloric acid) is added to dissolve the insoluble purple formazan product into a colored solution. The absorbance of this colored solution can be quantified by measuring at a certain wavelength (usually between 500 and 600 nm) by a spectrophotometer. The absorption maximum is dependent on the solvent employed.

MTS is a more recent alternative to MTT. MTS (3-(4,5-dimethylthiazol-2-yl)-5-(3-carboxymethoxyphenyl)-2-(4-sulfophenyl)-2H-tetrazolium), in the presence of phenazine methosulfate (PMS), produces a water-soluble formazan product that has an absorbance maximum at 490-500 nm in phosphate-buffered saline. It is advantageous over MTT in that (1) the reagents MTS+PMS are reduced more efficiently than MTT, and (2) the product is water soluble, decreasing toxicity to cells seen with an insoluble product. These reductions take place only when reductase enzymes are active, and therefore conversion is often used as a measure of viable (living) cells.

Proliferation assays are methods of measuring numbers of live cells. This may be better for some applications than measuring cell death or damage. For example, proliferation assays are able to reveal cytostatic, growth-inhibitory, and growth-enhancing effects which yield no readout in a cytotoxicity assay. Proliferation assays are also in common use as indirect cytotoxicity assays. Proliferation assays also fall into several categories. Certainly commonly used methods make use of tetrazolium salts, which are reduced in living cells to colored formazan dyes. One advantage of these methods is convenience, especially with the newer dyes (e.g., MTT and WST-1). The dye is added to the cell culture, and the absorbance of the formazan is read, typically after 0.5-12 hours.

It will also be recognized that cytotoxicity assays can be used as proliferation assays (and vice versa). To use a cytotoxicity assay to count live cells, one simply kills all the cells and performs the assay. (In some cases it may be necessary to wash the cells first, because the readout may depend on a molecule that may have been released into the supernatant by cells that have already died.)

One illustrative example of this approach is the ATP-release assay (see, e.g., Crouch et al. (1993) J. Immunol. Meth., 160: 81-88). Although strictly speaking this is a cytotoxicity assay, in that ATP released by dead cells is measured, it is rarely used as a direct cytotoxicity assay, because of the very short lifetime of extracellular ATP. Instead, the cells are killed with a lytic agent before the ATP is measured by the luciferase reaction. Thus even though the assay is basically a cytotoxicity assay, if it is to be used to measure cytotoxicity, it is an indirect method, like the other proliferation assays.

Another type of viability assay, also luminescent, is represented by a mitochondrion-based viability assay (Woods and Clements (2001) Nature Labscene UK March, 2001, 38-39).

An illustrative cytotoxicity assay based on release of alkaline phosphatase from target cells of killer lymphocytes was described by Kasatori et al. (1994) Rinsho Byori 42: 1050-1054).

A coupled luminescent method is described by Corey et al. (1997) J. Immunol. Meth. 207: 43-45). In this assay G3PDH activity is measured by coupling its cognate glycolytic reaction to the following reaction in glycolysis, which is carried out by phosphoglycerokinase (PGK). The PGK reaction produces ATP, which is then measured by luciferase, provided in a separate cocktail, yielding a luminance signal.

The foregoing assays are intended to be illustrative and not limiting. A number of other assays for cytotoxicity, and/or metabolic rate, and/or cell proliferation are known to those of skill in the art (see, e.g., Blumenthal (2005) Chemosensitivity: Volume I: In Vitro Assays (Methods in Molecular Medicine), Humana Press, New Jersey; U.S. Pat. No. 6,982,152, U.S. Patent Publication Nos: US 2005/0186557, US 2005/0112551 and PCT Publications: WO 2005/069000, WO 2003/089635, WO 2003/084333, WO 1994/006932, and the like).

In various embodiments the methods of screening agents for differential cytotoxicity (or differential protective activity) involve recording the identity of agents that are preferentially cytotoxic or protective to pluripotent stem cells (e.g., ESCs, iPSCs, etc.) over NSCs and/or preferentially cytotoxic or protective to pluripotent stem cells (e.g., ESCs, iPSCs, etc.) and/or NSCs over terminally differentiated cells in a database of agents that selectively inhibit the growth and/or proliferation of human pluripotent stem cells and/or neural stem cells. In certain embodiments the methods involve storing to a computer readable medium (e.g., an optical medium, a magnetic medium, a flash memory, etc.) the identity of agents that are preferentially cytotoxic or protective to pluripotent stem cells (e.g., ESCs, iPSCs, etc.) over NSCs and/or preferentially cytotoxic or protective to pluripotent stem cells (e.g., ESCs, iPSCs, etc.) and/or NSCs over terminally differentiated cells in a database of agents that to selectively inhibit the growth and/or proliferation of pluripotent stem cells (e.g., ESCs, iPSCs, etc.) and/or neural stem cells.

In certain embodiments the methods involve further screening said the selected agents for cytotoxic activity on cell lines. In various embodiments this involves contacting an embryonic stem cell and/or a neural stem cell (NSC) and/or a terminally differentiated cell with the test agent assaying the effect of that agent on cell metabolic activity, and/or proliferation, and/or cytotoxicity. In certain embodiments the terminally differentiated cell is a cell selected from the group consisting of a neuron, an astrocyte, and an oligodendrocyte.

In certain embodiments the agent is identified as an NSC killer if it shows cytotoxicity against NSCs with at least 1.5 fold or greater potency for NSCs than ESCs and shows at least a 25% reduction in viability of NSCs as compared to a control.

In certain embodiments the agent is identified as an NSC killer if it shows cytotoxicity against NSCs with at least 1.5 fold or greater potency for NSCs than ESCs and shows at least a 25% reduction in viability of NSCs as compared to a control.

In certain embodiments the agent is identified as an NSC killer if it reduces ATP concentrations with at least 2-fold or more potency for NSCs than ESCs, and that NSC values are 50% or more below a control mean.

In certain embodiments the agent is identified as an ESC killer if there is any significant selectivity for affecting ATP levels in ESCs over NSCs.

### Methods of Generating Substantially Homogenous Populations of Cells.

In certain embodiments, using the screening methods described herein, compounds are identified that are toxic to neural stem cells (NSCs), but not to embryonic stem cells (ESCs) or that show greater toxicity against NSCs than ESCs (see Tables 1 and 2). These compounds can be used to prepare substantially homogenous populations of ESCs. Conversely, compounds are also identified herein that show greater toxicity to ESCs than to NSCs and can be used, for example, to generate substantially homogeneous populations of NSCs.

The screening methods described herein have bee used to identified FDA-approved drugs that can specifically or preferentially kill either hESCs or NSC or both. Compounds showing such differential toxicity obtained from the National Institutes of Neurological Disorders and Stroke (NINDS) compound library are shown in Table 1. Compounds showing such differential toxicity obtained from the PRESTWICK CHEMICAL LIBRARY® are shown in Table 2.

TABLE 1
NINDS screening data.

| | ESC/NSC |
|---|---|
| Compounds Toxic to NSCs not ESCs | |
| Clofoctol | 1135.9962 |
| Selamectin | 988.925399 |
| Hexetidine | 836.776807 |
| Amiodarone Hydrochloride | 819.533152 |
| Flunarizine Hydrochloride | 229.871967 |
| Chloroacetoxyquinoline | 176.350281 |
| Menadione | 118.316186 |
| Gossypol-Acetic Acid Complex | 13.6531157 |
| Promazine Hydrochloride | 11.9330921 |
| Cytarabine | 11.2142124 |
| Meclizine Hydrochloride | 8.29714363 |
| Fenbendazole | 7.31923358 |
| Nigericin Sodium | 7.18157402 |
| Thioguanine | 6.77287707 |
| Perhexilline Maleate | 6.70634586 |
| Azaserine | 6.28070036 |
| Mycophenolic Acid | 5.03719947 |
| Levodopa | 4.81057989 |
| Methotrexate | 4.72778023 |
| Bromhexine Hydrochloride | 3.68991875 |
| OLIGOMYCIN (A Shown) | 3.34368839 |
| Eburnamonine | 3.03091179 |
| Emetine Hydrochloride | 2.54728406 |
| Edoxudine | 2.41546874 |
| Tamoxifen Citrate | 2.29293769 |
| Toxic to both but are (>5x) more toxic to NSCs | |
| Cloxyquin | 534.330052 |
| Calcimycin | 377.638425 |
| Puromycin Hydrochloride | 247.252105 |
| Gentian Violet | 210.391726 |
| Thimerosal | 165.610322 |
| Pyrithione Zinc | 116.370342 |
| Tyrothricin | 109.433931 |
| Cetylpyridinium Chloride | 109.408014 |
| Pyrvinium Pamoate | 85.9103544 |
| Pararosaniline Pamoate | 62.8645407 |
| Phenylmercuric Acetate | 25.2666319 |
| Sanguinarine Nitrate | 8.4456945 |
| Floxuridine | 8.4456945 |
| Mitoxanthrone Hydrochloride | 6.73983865 |
| Nerifolin | 6.69591256 |
| Patulin | 6.25503134 |
| Cetrimonium Bromide | 5.40223398 |
| Quinacrine Hydrochloride | 5.14302071 |
| Anisomycin | 5.12350911 |
| Acriflavinium Hydrochloride | 5.03653792 |

TABLE 2
Prestwick screening data.

| Toxic To NSC (and not ESCs) | ESC/NSC |
|---|---|
| Amethopterin (R,S) | 6.45036371 |
| Methiazole | 3.00981909 |
| Trifluridine | 2.95542685 |
| Bisacodyl | 2.70096279 |
| Lasalocid Sodium Salt | 2.05164122 |
| Pyrimethamine | 2.00883563 |
| Chelidonine (+) | 1.99858969 |
| Toxic to both but (>5x) more toxic to NSCs | ESC/NSC |
| Cantharidin | 6.17111032 |
| Tomatine | 9.24232343 |
| Sanguinarine | 102.588026 |
| Toxic To ESCs (and not NSCs) | NSC/ESC |
| Disulfiram | 10.2968346 |
| Beta-Belladonnine Dichloroethylate | 2.71741828 |
| (D,L)-Tetrahydroberberine | 2.33648787 |
| Flurandrenolide | 2.30824868 |
| Parthenolide | 2.21588177 |
| Clofilium Tosylate | 2.18374832 |
| Sulfamerazine | 2.00290316 |
| Zardaverine | 1.97597438 |
| Fluticasone Propionate | 1.95378917 |
| Nitrarine Dihydrochloride | 1.949293 |
| Pyrilamine Maleate | 1.93369289 |
| Gbr 12909 Dihydrochloride | 1.75366025 |
| (−)-Levobunolol Hydrochloride | 1.68916275 |
| Toxic to both but (5x) more toxic to ESCs | NSC/ESC |
| Camptothecine (S,+) | 14.4618855 |
| Puromycin Dihydrochloride | 10.8444565 |
| Doxorubicin Hydrochloride | 8.90614084 |
| Paclitaxel | 5.32536807 |

One or more of the compounds listed in Tables 1 and 2 can be used to generate substantially homogenous populations of embryonic stem cells, neural stem cells, or terminally differentiated cells.

Method of Generating a Substantially Homogenous Population of Pluripotent Stem Cells (e.g., ESCs, iPSCs, etc.).

Accordingly, in certain embodiments, methods are provided for generating a substantially homogenous population of pluripotent stem cells (e.g., ESCs, iPSCs, etc.). In various embodiments the methods involve providing a population of pluripotent stem cells (e.g., ESCs, and/or iPSCs, etc.) and contacting the population with one or more agent(s) that preferentially kill progenitor cells (e.g., NSCs). In certain embodiments the agent(s) are provided in an amount to preferentially kill NSCs while leaving viable embryonic stem cells, and in certain embodiments, without substantially diminishing the population and/or viability of embryonic stem cells. In certain embodiments the agent(s) are selected from the group consisting of amethopterin (r,s), methiazole, trifluridine, bisacodyl, lasalocid sodium salt, pyrimethamine, chelidonine (+), cantharidin, tomatine, sanguinarine, clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin, eburnamonine, emetine hydrochloride, edoxudine, tamoxifen citrate, cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, and acriflavinium hydrochloride.

In certain embodiments the agent(s) are selected from the group consisting of amethopterin (r,s), methiazole, trifluridine, bisacodyl, lasalocid sodium salt, pyrimethamine, chelidonine (+), clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin, eburnamonine, emetine hydrochloride, edoxudine, tamoxifen citrate.

Method of Generating a Substantially Homogenous Population of Adult Stem Cells (e.g., NSCs).

In certain embodiments methods are provided for generating a substantially homogenous population of adult stem cells derived from pluripotent stem cells (e.g., hESCs, iPSCs, etc.). In various embodiments the method involves differentiating adult stem cells from a population of pluripotent stem cells (e.g., hESCs) to form a population of adult stem cells (or simply providing a population of adult stem cells (e.g., from a commercial supplier)); and contacting the population with one or more agent(s) that preferentially inhibit the growth or proliferation of human embryonic stem cells remaining in said population, thereby producing a substantially homogenous population of adult stem cells. In various embodiments the adult stem cells are neural stem cells (NSCs).

In various embodiments the agent(s) comprise one or more compounds selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, gbr 12909 dihydrochloride, (−)-levobunolol hydrochloride, camptothecine (s,+), puromycin dihydrochloride, doxorubicin hydrochloride, and paclitaxel.

In various embodiments the agent(s) comprise one or more compounds selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, gbr 12909 dihydrochloride, and (−)-levobunolol hydrochloride.

In various embodiments the population of differentiated cells comprises a population of postmitotic neuron cells.

Methods of Generating a Substantially Homogenous Differentiated Population of Cells Derived from Pluripotent Stem Cells (e.g., hESCs, iPSCs, etc.)

In certain embodiments methods are provided for generating a substantially homogenous population of differentiated cells (e.g., terminally differentiated) derived from pluripotent stem cells (e.g., hESCs, iPSCs, etc.). In various embodiments the method involves differentiating cells from a population of pluripotent stem cells to form a population of differentiated cells (or simply providing a population of differentiated cells (e.g., from a commercial supplier)); and contacting the population with one or more agents that preferentially inhibit the growth or proliferation of pluripotent stem cells and/or adult stem cells in the population, thereby producing a substantially homogenous differentiated population of cells. In certain embodiments the population of differentiated cells comprises a population of differentiated neural cells (e.g., neurons, astrocytes, oligodendrocytes, etc.).

In certain embodiments the contacting comprises contacting the population with one or more agents that are toxic to both pluripotent stem cells (e.g., hESCs, iPSCs, etc.) and NSCs and the agent(s) are selected from the group consisting of cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, acriflavinium hydrochloride, cantharidin, tomatine, sanguinarine, camptothecine (s,+), puromycin dihydrochloride, doxorubicin hydrochloride, and paclitaxel.

In certain embodiments the contacting comprises contacting the population with one or more agent(s) that are toxic to pluripotent stem cells (e.g., hESCs, iPSCs, etc.) where the agent(s) are selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, GBR 12909 dihydrochloride, (−)-levobunolol hydrochloride; and an agent that is toxic to NSCs or to both NSCs and pluripotent stem cells, where the agent(s) toxic to NSCs are selected from the group consisting of clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin (a shown), eburnamonine, emetine hydrochloride, edoxudine, tamoxifen citrate, amethopterin (r,s), methiazole, trifluridine, bisacodyl, lasalocid sodium salt, pyrimethamine, and chelidonine (+), and the agent(s) toxic to both NSCs and ESCs are selected from the group consisting of cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, acriflavinium hydrochloride, cantharidin, tomatine, sanguinarine, camptothecine (s,+), puromycin dihydrochloride, doxorubicin hydrochloride, and paclitaxel.

In certain embodiments the contacting comprises contacting the population with: one or more agent(s) that is toxic to NSCs where the agent(s) are selected from the group consisting of clofoctol, selamectin, hexetidine, amiodarone hydrochloride, flunarizine hydrochloride, chloroacetoxyquinoline, menadione, gossypol-acetic acid complex, promazine hydrochloride, cytarabine, meclizine hydrochloride, fenbendazole, nigericin sodium, thioguanine, perhexylline maleate, azaserine, mycophenolic acid, levodopa, methotrexate, bromhexine hydrochloride, oligomycin (a shown), eburnamonine, emetine hydrochloride, edoxudine, tamoxifen citrate, amethopterin (r,s), methiazole, trifluridine, bisacodyl, lasalocid sodium salt, and pyrimethamine, chelidonine (+); and one or more agent(s) that are toxic to ESCs or to both NSCs and ESCs, where the agent(s) toxic ESCs where the agent are selected from the group consisting of disulfuram, beta-belladonnine dichloroethylate, (d,l)-tetrahydroberberine, flurandrenolide, parthenolide, clofilium tosylate, sulfamerazine, zardaverine, fluticasone propionate, nitrarine dihydrochloride, pyrilamine maleate, GBR 12909 dihydrochloride, and (−)-levobunolol hydrochloride, and the agent toxic to both NSCs and ESCs is selected from the group consisting of cloxyquin, calcimycin, puromycin hydrochloride, gentian violet, thimerosal, pyrithione zinc, tyrothricin, cetylpyridinium chloride, pyrvinium pamoate, pararosaniline pamoate, phenylmercuric acetate, sanguinarine nitrate, floxuridine, mitoxanthrone hydrochloride, nerifolin, patulin, cetrimonium bromide, quinacrine hydrochloride, anisomycin, acriflavinium hydrochloride, cantharidin, tomatine, sanguinarine, camptothecine (s,+), puromycin dihydrochloride, doxorubicin hydrochloride, and paclitaxel.

In certain embodiments, where the agent(s) are selected from the group consisting of selamectin, amiodarone HCL, and minocycline HCL, and an analogue thereof.

### High Throughput Screening

Any of the assays described herein are amenable to high-throughput screening (HTS). Moreover, the cells utilized in the methods of this invention need not be contacted with a single test agent at a time. To the contrary, in certain embodiments, to facilitate high-throughput screening, a single cell may be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for various measures of metabolic activity and/or cytotoxicity are well known to those of skill in the art. For example, multi-well fluorimeters are commercially available (e.g., from Perkin-Elmer).

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting cytotoxicity markers, ATP assays, and the like.

### Candidate Agent Databases.

In certain embodiments, the agents that score positively in the assays described herein (e.g., show differential activity against pluripotent stem cells and adult stem cells na/dor progenitor cells) can be entered into a database of putative and/or actual agents to show differential cytotoxic or protective activity against, for example, pluripotent stem cells (e.g., ESCs, iPSCs, etc.) and adult stem cells (e.g., NSCs). The term database refers to a means for recording and retrieving information. In certain embodiments the database also provides means for sorting and/or searching the stored information. The database can comprise any convenient media including, but not limited to, paper systems, card systems, mechanical systems, electronic systems, optical systems, magnetic systems or combinations thereof. Typical databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

### Kits.

In another embodiment, this invention provides kits for the screening procedures and/or the culture methods described herein. In various embodiments, the kits one or more of the following: pluripotent stem cells (e.g., ESCs, and/or iPSCs, etc.), adult stem cells, NSCs, one or more of the compounds listed in Tables 1 or 2, and the like.

In addition, the kits optionally include labeling and/or instructional materials providing directions (i.e., protocols) for the practice of the culture methods and/or screening methods described herein. In certain embodiments instructions materials describe methods of identifying agents that show differential cytotoxicity or protective activity on ESCs and NSCs, and/or teach methods of generating substantially homogenous populations of ESCs, NSCs, and/or terminally differentiated cells. In various embodiments the instructions materials teach the use of one or more compounds listed in Tables 1 and 2 in the methods described herein.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Identification by Automated Screening of a Small Molecule that Selectively Eliminates Neural Stem Cells Derived from hESCs, but not hESC-Derived Dopaminergic Neurons

In this example, we tested the hypothesis that a differential screen using, for example, US Food and Drug Administration (FDA)-approved compounds can identify compounds that either selective survival factors or specific toxins and may be useful for the therapeutically-driven manufacturing of cells in vitro and possibly in vivo.

We designed a set of experiments aimed at developing a hESC-based automated assay for screening small molecules that have differential toxicity to hESC-derived NSCs and their differentiated neural progenies. We reasoned that the development of this assay would help identify chemical compounds that may be useful for eliminating proliferating cells in potential hESC-derived cell therapy products. To this end, we chose to use the National Institute of Neurodegenerative Diseases and Stroke (NINDS) collection of FDA-approved drugs for assay optimization and pilot screening. The bioactivity of the compounds in this library and the ready availability of individual compounds identified as hits for follow-up studies make this library ideal for pilot screenings. Furthermore, these routinely used drugs have been highly optimized to hit specific targets and in nearly all cases the mechanisms of action are known.

By comparative screening on hESCs and hESC-derived homogenous NSCs using the NINDS collection, we were able to identify compounds that had differential toxicity to both cell populations. Hits obtained in the primary screen were then retested and a small subset was assayed for dose-responsiveness. One confirmed dose-responsive compound, amiodarone HCl, was further tested for toxicity in postmitotic neurons. We found amiodarone HCL to be toxic to NSCs but not to postmitotic neurons, indicating its potential use for depleting proliferating NSCs in hESC-derived cell populations for possible neural transplantation.

### Materials and Methods

Culturing of hESCs and hESC-Derived NSCs

hESC lines I6 and H9 were maintained on Matrigel (BD Biosciences, Bedford, Mass.; www.bdbiosciences.com) coated dishes in medium (comprised of Dulbecco's Modified Eagle's Medium/Ham's F12 supplemented with 20% knockout serum replacement (KSR), 2 mM non-essential amino acids, 4 mM L-glutamine, 0.1 mM β-mercaptoethanol, 50 mg/ml Penn-Strep, and 4 ng/ml of basic fibroblast growth factor) conditioned with mouse embryonic fibroblasts for 24 hours as previously described (Cai J, Chen J, Liu Y, Miura T, Luo Y, et al. (2005) Assessing self-renewal and differentiation in hESC lines. Stem Cells; Schulz et al. (2007) BMC Genomics 8: 478).

To derive NSCs as previously described (Swistowski et al. (2009) PLoS One 4: e6233), hESC colonies were harvested using a scraper and cultured in suspension as EBs for 8 days in ESC medium minus FGF2. EBs were then cultured for additional 2-3 days in suspension in neural induction media containing DMEM/F12 with Glutamax, 1×NEAA, 1×N2 and FGF2 (20 ng/ml) prior to attachment on cell culture plates. Numerous neural rosettes were formed 2-3 days after adherent culture. To obtain a pure population of NSCs, rosettes were manually isolated and dissociated into single cells using Accutase. The NSCs population was expanded in Neurobasal media containing 1×NEAA, 1×L-Glutamine (2 mM), 1×B27, LIF and FGF2 20 ng/ml.

Dopaminergic neuronal differentiation of hESC-derived NSCs was induced by medium conditioned on the PA6 stromal cell line for 4 weeks. The media contained GMEM with 10% KSR, 1× nonessential AA, 1× Na pyruvate and 1× β-mercaptoethanol and was harvested from the PA6 culture every 24 h for a period of 1 week.

Human astrocytes were purchased from Sciencell Research Laboratories (isolated from human cerebral cortex, Cat#1800, Carlsbad, Calif.) and were cultured in human astrocyte medium (Sciencell, Cat#1801) on poly-L-lysine coated tissue culture dishes. Media was changed every other day and cells were passaged once a week at a 1:4 ratio.

2102Ep cells, derived from a primary human testicular teratocarcinoma and later subcloned (Andrews et al. (1982) Int J Cancer 29: 523-531) (ATCC) were grown on tissue culture dishes in medium containing DMEM supplemented with 2 mM Glutamax and 10% fetal bovine serum. Media was changed every day and cells were passaged every 3-4 days at a ratio of between 1:4 to 1:6.

Drug Treatment and ATP Assay

hESCs and NSCs were passaged onto 96 well plates at a density of 56104 and 2.66104 cells respectively in 200 ml media and incubated at 37° C. for 48 hours. Media was changed every day for hESCs and every other day for NSCs and additionally changed prior to drug treatment. The cells were treated with compounds from the NINDS library diluted in 100 ml of either ESC or NSC media to a final concentration of 2.5 mM in 0.01% DMSO. Cells were incubated in the presence of drug for an additional 48 hours at 37° C. before assaying. For all sampling, ESC and NSC plates were processed in parallel for one drug or control condition at a time.

For ATP measurements, the media was removed, cells were washed 1× in milliQ water and reconstituted in 50 mL ATP-Lite Mammalian Lysis Buffer and shaken for 5 minutes. Two 10 mL aliquots of lysed cells were replated onto separate 96 well plates for later protein measurements.

For measuring the effect of TNFc on NSCs, 16 NSCs were passaged onto fibronectin-coated 4-well plates in Neurobasal media supplemented with 1×B27, 2 mM L-glutamine and 10 ng/ml of both bFGF and LIF growth factors. Cells were recovered for 12 hours at 37° and then either left untreated or treated with solTNFα at the concentrations indicated. Cultures were observed for 24 hours after solTNFc treatment for signs of cell death and imaged with microscopy.

Immunocytochemistry

Immunocytochemistry and staining procedures were as described previously (Zeng et al. (2003) Stem Cells 21: 647-653). Briefly, hESCs at different stages of dopaminergic differentiation were fixed with 2% paraformaldehyde for half an hour. Fixed cells were blocked for one hour in 0.1% Triton X-100 PBS supplemented with 10% goat serum and 1% BSA, followed by incubation with the primary antibody at 4° C. overnight in 0.1% Triton X-100 with 8% goat serum and 1% BSA. Appropriately coupled secondary antibodies (Molecular Probes) were used for single and double labeling. All secondary antibodies were tested for cross reactivity and non-specific binding. The following primary antibodies were used: Oct-4 (19857 Abcam) 1:1000; 3411 tubulin clone SDL.3D10 (T8660 Sigma) 1:500; Nestin (611658 BD Transduction laboratories) 1:500 and TH (P40101 Pel-Freez) 1:500, and as secondary antibodies: Alexa Fluor 594 Goat Anti-Mouse, Alexa Fluor 488 Goat Anti-Rabbit, Alexa Fluor 594 Goat Anti-Rabbit. Hoechst 33342 (Molecular Probes H3570) 1:5000 was used for nuclei identification. Images were captured on a Nikon fluorescence microscope.

Microarray Analysis Using BeadArray Platform

RNAs isolated from NSCs and neurons with and without drug treatments were hybridized to Illumina HumanRef-8 BeadChip (Illumina, Inc., San Diego, Calif., performed by Microarray core facility at the Burnham Institute for Medical Research). The Illumina array data were normalized by the quantile method, and then transformed log 2 ratio values for a zero mean for expression values of each gene across all samples. The statistical and bioinformatics analyses were conducted by using R and the bioconductor package (www.bioconductor.org). The gene set enrichment analysis was conducted using the GSEA software (www.broad.mit.edu/gsea).

### Results

Culturing of Multiple hESC and hESC-Derived NSC Lines in 96-Well Plates

We have shown that NSCs can be generated from multiple hESC lines and can be cultured for prolonged periods without losing their ability to differentiate into neurons, astrocytes and oligodendrocytes (Swistowski et al. (2009) PLoS One 4: e6233). The hESC lines H9 and 16 and their NSC derivatives behave similarly in culture and were used for this study.

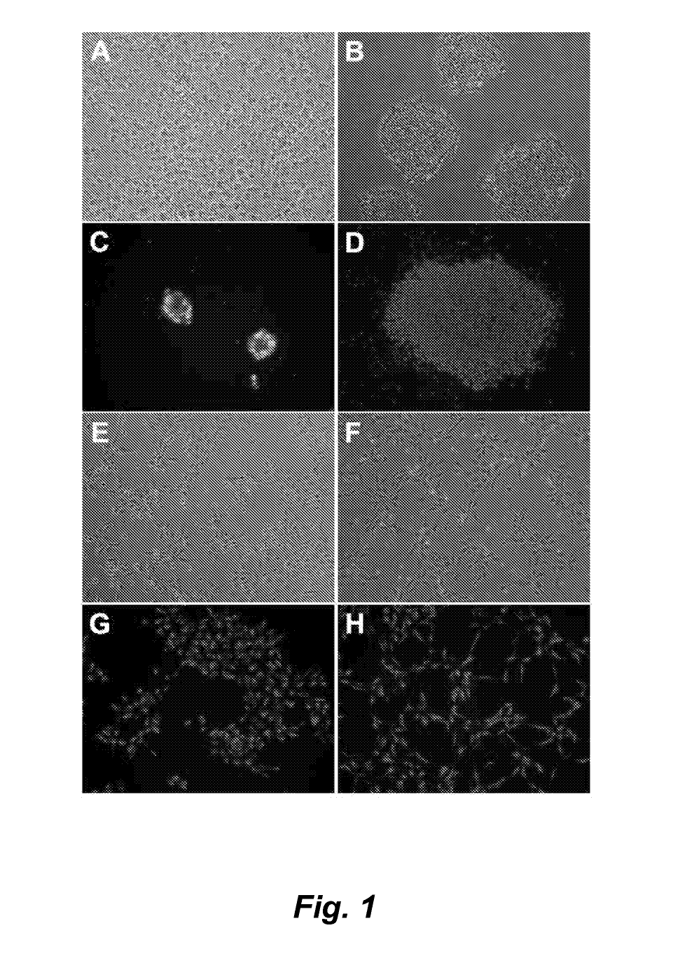

For adapting to a 96-well format culture, hESCs were dissociated into single cells by Accutase. Tiny colonies were formed 24 h after plating (FIG. 1, panel A) and typical undifferentiated hESC morphology was observed 2-3 days after passage (FIG. 1, panel B).

No differences in the expression of the pluripotent marker Oct4 (FIG. 1, panels C-D) were found between cells cultured in 96-well plates and hESCs routinely passaged in medium conditioned on MEF in larger dishes (35-mm or 60-mm dishes). NSCs cultured in 96-well plates were morphologically indistinguishable from cells cultured in larger dishes (FIG. 1, panels E-F) and uniformly expressed the NSC specific marker Nestin (FIG. 1, panels G-H).

Screening Design, Primary Screening and Retest of Hits

To identify compounds that are toxic to hESCs, hESC-derived NSCs, or both, we screened 720 FDA-approved drugs of the NINDS collection by testing the toxicity of each drug at a dose of 2.5 mM. For endpoint measurement of cell death caused by drug toxicity, we used a widely accepted ATP assay that measures changes in ATP level as an indicator of cellular response to cell death. In this assay, total ATP content per well was measured and normalized to the total cellular protein.

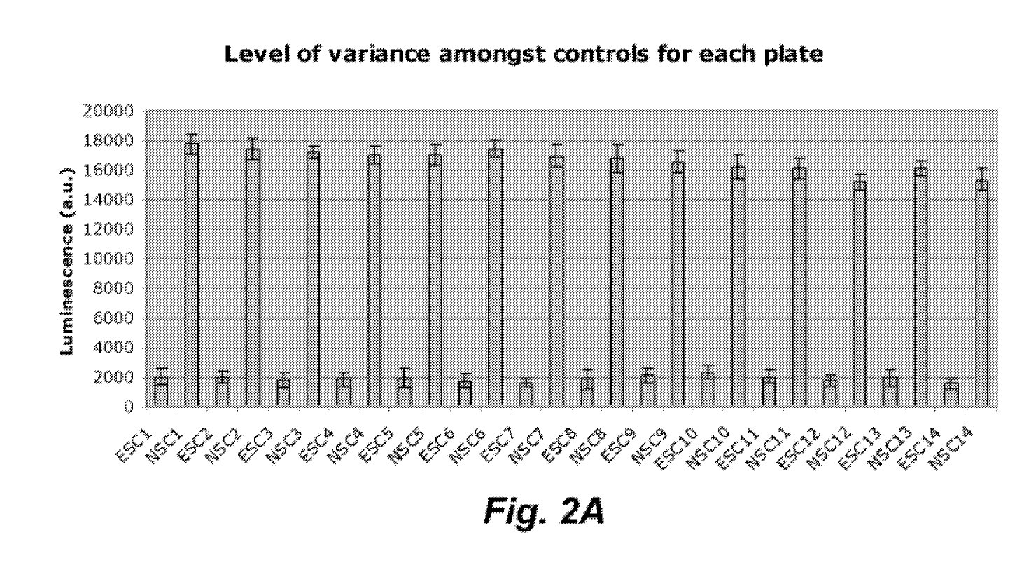

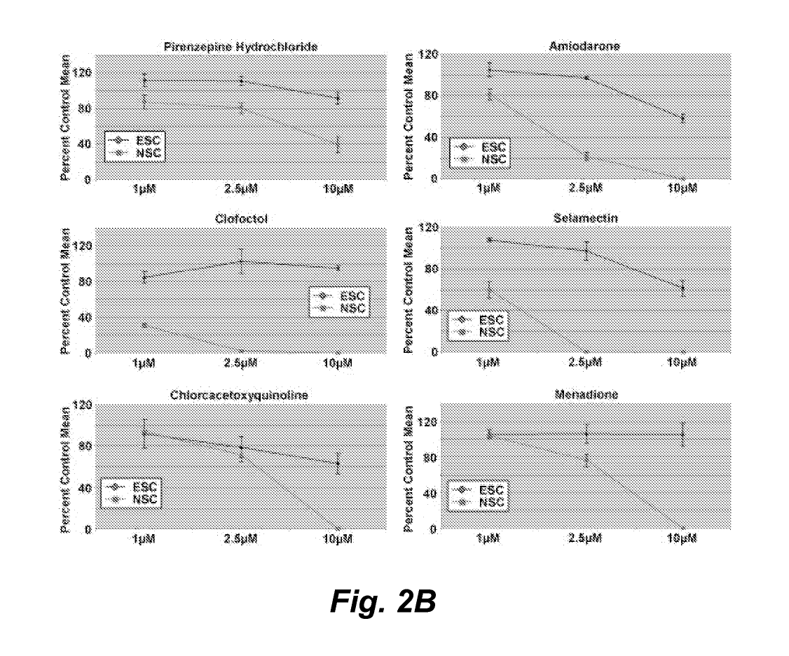

In general, NSC-containing wells had much higher ATP levels than the hESC wells (FIG. 2, panel A, standard deviation for variance in each plate provided in Table 3), consistent with recent reports that ATP levels are higher in differentiated EBs than in undifferentiated hESCs (Cho et al. (2006) Biochem Biophys Res Commun 348: 1472-1478). Hits were defined based on the ability of a compound to affect ATP levels relative to DMSO controls on each plate. Nine compounds, pirenzepine HCL, amiodarone HCL, selamectin, clofoctol, perhexylline maleate, griseofulvin, chloroactoxyquinoline, menadione and hexetidine were identified as “NSC Killers” in this primary screen. Application of these nine drugs reduced ATP concentrations with at least 2-fold or more potency for NSCs than hESCs, and NSC values were 15% or more below the control mean. In contrast, no compound was found to be specifically toxic to hESCs based on the same criteria.

TABLE 3
Controls for FIG. 2.
Table S4: Controls for FIG. 2
DMSO Controls for each plate:

| | ESC1 | NSC1 | ESC2 | NSC2 | ESC3 | NSC3 | ESC4 |
|---|---|---|---|---|---|---|---|
| A1 | 1755 | 17723 | 1634 | 17785 | 1942 | 17330 | 2234 |
| A12 | 1819 | 17126 | 2340 | 17941 | 802 | 17847 | 1891 |
| B1 | 1652 | 16896 | 2321 | 17264 | 1914 | 17272 | 1660 |
| B12 | 2265 | 16849 | 1828 | 17337 | 1279 | 16886 | 1175 |
| C1 | 2968 | 17930 | 2771 | 17180 | 2327 | 16561 | 2175 |
| C12 | 1596 | 18111 | 1931 | 18188 | 1633 | 17291 | 1345 |
| D1 | 2571 | 18941 | 2079 | 18180 | 1753 | 17133 | 1545 |
| D12 | 2698 | 17098 | 1728 | 17455 | 1955 | 17477 | 2040 |
| E1 | 1331 | 17236 | 2524 | 18065 | 2410 | 17411 | 2689 |
| E12 | 1488 | 17156 | 1510 | 17129 | 2085 | 17933 | 1515 |
| F1 | 2760 | 19031 | 1873 | 15669 | 2653 | 17667 | 2598 |
| F12 | 1913 | 18164 | 1893 | 18149 | 1999 | 16854 | 1505 |
| G1 | 2595 | 18380 | 2022 | 17411 | 1284 | 17306 | 2166 |
| G12 | 2110 | 17684 | 2329 | 17037 | 1424 | 16918 | 1681 |
| H1 | 1543 | 18344 | 1642 | 16255 | 1424 | 16553 | 2225 |
| H12 | 1511 | 17393 | 1660 | 17680 | 1765 | 16830 | 1371 |
| | ESC8 | NSC8 | ESC9 | NSC9 | ESC10 | NSC10 | ESC11 |
| A1 | 1629 | 16451 | 2165 | 17040 | 2568 | 16904 | 2250 |
| A12 | 1435 | 15916 | 2201 | 17153 | 2164 | 14263 | 1632 |
| B1 | 2661 | 18197 | 2119 | 17699 | 1943 | 17220 | 2046 |
| B12 | 1224 | 15837 | 2140 | 16368 | 2001 | 15533 | 1862 |
| C1 | 2255 | 17544 | 2286 | 16897 | 2433 | 16624 | 1532 |
| C12 | 1164 | 16207 | 1490 | 15922 | 2053 | 16418 | 1325 |
| D1 | 1783 | 18506 | 1934 | 16920 | 2914 | 16824 | 2588 |
| D12 | 1497 | 16373 | 2995 | 16522 | 2076 | 16456 | 1750 |
| E1 | 1767 | 17880 | 2684 | 17171 | 3597 | 16801 | 1920 |
| E12 | 1716 | 16023 | 2560 | 17316 | 2139 | 15907 | 2305 |
| F1 | 2301 | 17812 | 1116 | 16289 | 2971 | 16703 | 2394 |
| F12 | 1441 | 16072 | 1956 | 15945 | 2098 | 14804 | 1648 |
| G1 | 2118 | 17419 | 1655 | 16754 | 1991 | 16350 | 2256 |
| G12 | 1596 | 15972 | 2737 | 14998 | 2581 | 15384 | 2092 |
| H1 | 3602 | 16193 | 1467 | 16389 | 1726 | 16488 | 2891 |
| H12 | 1640 | 15815 | 1606 | 15261 | 2122 | 16233 | 2254 |

| Control | E1 | N1 | E2 | N2 | E3 | N3 | E4 |
|---|---|---|---|---|---|---|---|
| avg | 2035.938 | 17753.88 | 2005.313 | 17420.31 | 1790.563 | 17204.31 | 1863.438 |
| Stdev | 534.5344 | 695.1363 | 361.1001 | 699.1367 | 476.2212 | 416.549 | 456.1364 |
| Control | E8 | N8 | E9 | N9 | E10 | N10 | E11 |
| avg | 1864.313 | 16763.56 | 2069.438 | 16540.25 | 2336.063 | 16182 | 2046.563 |
| Stdev | 615.1734 | 948.3536 | 518.1304 | 737.2153 | 484.9061 | 809.5746 | 414.5881 |

| NSC4 | ESC5 | NSC5 | ESC6 | NSC6 | ESC7 | NSC7 |
|---|---|---|---|---|---|---|
| 17021 | 1126 | 16840 | 1909 | 17436 | 1746 | 16512 |
| 16178 | 1962 | 16333 | 2150 | 17207 | 1491 | 16961 |
| 16886 | 1621 | 17076 | 2140 | 17297 | 1798 | 16092 |
| 16832 | 1519 | 17623 | 1725 | 16133 | 1162 | 17121 |
| 17676 | 1720 | 17088 | 1491 | 18154 | 1368 | 17554 |
| 17600 | 1880 | 17711 | 2257 | 17334 | 1736 | 17832 |
| 17827 | 1990 | 17167 | 1912 | 17394 | 1886 | 16530 |
| 17008 | 1355 | 17211 | 659 | 17928 | 1576 | 17600 |
| 17556 | 4129 | 18612 | 1937 | 17991 | 1634 | 17350 |
| 16785 | 1276 | 16707 | 1523 | 17549 | 1058 | 17119 |
| 16789 | 1954 | 17117 | 1951 | 18196 | 1777 | 17304 |
| 16965 | 1984 | 16767 | 1180 | 17331 | 1423 | 16586 |
| 18076 | 1981 | 16072 | 1448 | 17774 | 1762 | 17850 |
| 16874 | 2332 | 16995 | 1458 | 16675 | 1757 | 16277 |
| 16460 | 2044 | 16695 | 2172 | 17446 | 1882 | 17522 |
| 15875 | 2145 | 15690 | 1808 | 17286 | 1690 | 14871 |
| NSC11 | ESC12 | NSC12 | ESC13 | NSC13 | ESC14 | NSC14 |
| 16569 | 1623 | 15544 | 1915 | 17013 | 2488 | 16986 |
| 16580 | 1829 | 14886 | 2083 | 14879 | 1510 | 14152 |
| 16469 | 2136 | 14755 | 2741 | 16623 | 1765 | 16036 |
| 15520 | 1594 | 15440 | 1854 | 15777 | 1213 | 14601 |
| 16337 | 1500 | 16253 | 1410 | 16274 | 1663 | 15426 |
| 15372 | 1488 | 15393 | 1389 | 15725 | 1012 | 15186 |
| 16526 | 2393 | 15542 | 1756 | 16329 | 1908 | 15559 |
| 16777 | 1823 | 15146 | 1101 | 15851 | 1540 | 15341 |
| 16408 | 2029 | 14992 | 2587 | 16585 | 1796 | 15244 |
| 15340 | 1495 | 15102 | 1254 | 15455 | 1128 | 13943 |
| 17086 | 1958 | 15093 | 2995 | 16361 | 1585 | 15908 |
| 14419 | 1386 | 14595 | 1908 | 15955 | 1449 | 14708 |
| 16527 | 1585 | 14233 | 2064 | 16222 | 1873 | 15104 |
| 15843 | 1410 | 14176 | 1732 | 16017 | 1190 | 15133 |
| 16010 | 1759 | 15721 | 2249 | 16596 | 1497 | 16006 |
| 15247 | 2457 | 15708 | 2168 | 16110 | 1417 | 15635 |

| Control | N4 | E5 | N5 | E6 | N6 | E7 | N7 |
|---|---|---|---|---|---|---|---|
| avg | 17025.5 | 1938.625 | 16981.5 | 1732.5 | 17445.69 | 1609.125 | 16942.56 |
| Stdev | 596.6514 | 672.3943 | 676.6239 | 422.9638 | 526.8276 | 246.8062 | 777.0393 |
| Control | N11 | E12 | N12 | E13 | N13 | E14 | N14 |
| avg | 16064.38 | 1779.063 | 15161.19 | 1950.375 | 16110.75 | 1564.625 | 15310.5 |
| Stdev | 711.0125 | 336.0051 | 557.2495 | 529.1494 | 516.0134 | 362.6519 | 754.0268 |

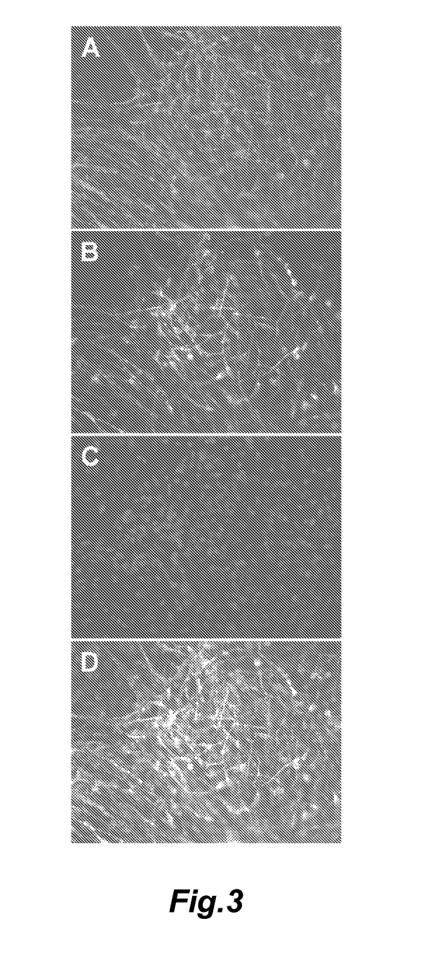

P value for Amidarone-10 uM <0.001
FIG. 3
10 uM ESC vs DA 0.0013
10 uM NSC vs DA <0.001
10 uM ESC vs NSC <0.001

We then retested the nine hits from the NINDS library screening in 96-well plates. Three concentrations of each compound (1 mM, 2.5 mM and 10 mM) were used in the retest. Six of the nine compounds, amiodarone HCL, selamectin, chloroacetoxyquinoline, menadione, pirenzepene and clofoctol showed a dose-dependent specific toxicity as demonstrated by reduced ATP concentrations in treated NSCs versus untreated NSCs, untreated hESCs and treated hESCs (FIG. 2, panel B). Notably, of these 6 compounds that demonstrated dose responsive toxicity to NSCs, selamectin and amiodarone HCL had the most dramatic effect on NSC survival (FIG. 2, panel B, p<0.001 for amiodarone HCL treated NSC versus similarly treated ESC, N=3 independent replicates). Overall, these results indicate that changes in ATP levels are a reliable indicator of cell death in stem cell populations upon drug insults and may have utility for hESC-based automatic screening assays.

Revalidation in Larger Numbers of Cells and Behavior of a Candidate Molecule on Postmitotic Neurons

For potential hESC-based neural replacement therapy, it would be useful to identify compounds that are selectively toxic to proliferating NSCs and not terminally differentiated postmitotic neurons. We therefore decided to interrogate the effects of one retested compound, amiodarone HCl, on NSCs and their differentiated derivatives. For postmitotic neurons, we chose to use an established neuronal differentiation culture system in which NSCs were induced to differentiate into dopaminergic neurons by medium conditioned on stromal cells for 4 weeks. After 4 weeks of differentiation, the majority of the cells (0.60%) expressed the postmitotic neuronal marker 3-111 tubulin with a subset (about 50% of total neurons) additionally expressing TH, a marker for midbrain dopaminergic neurons (FIG. 3, panels A, D). Less than 1% of the cells were positively stained for Sox1, a marker for NSCs (data not shown). Cells at this stage are referred to as dopaminergic neurons in this study.

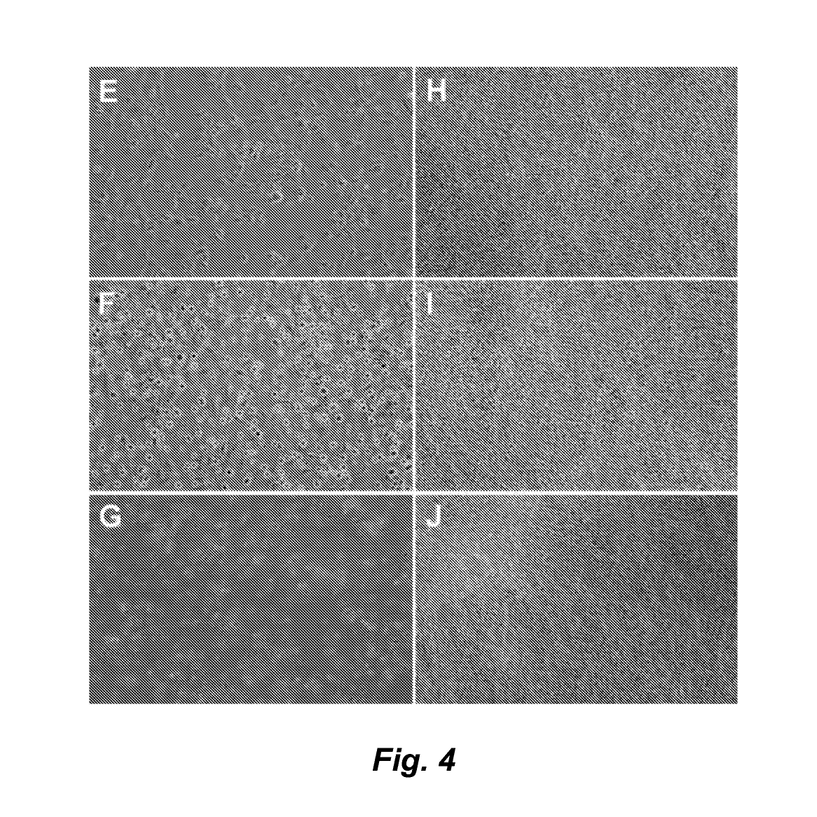

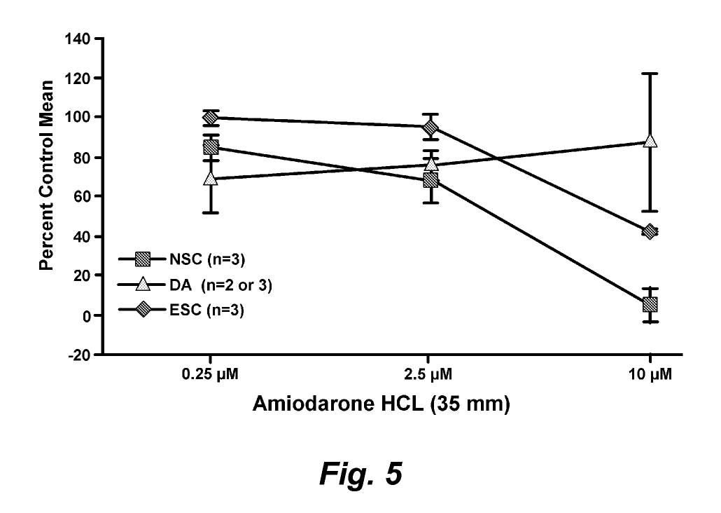

NSCs and dopaminergic neurons grown in 35-mm dishes were exposed to amiodarone HCl. Cell death was observed in NSCs 2 hours after drug exposure, with more than 90% cell death evident by 8 hours (FIG. 4, panels E, G). In contrast, no toxic effect was observed in dopaminergic neurons up to 8 hours after exposure to amiodarone HCl (FIG. 4, panels H-J) at the highest dose (10 mM). At 10 mM, amiodarone HCL reduced ATP levels to less than 15% of the control mean specifically in the NSC population (FIG. 5). In contrast, at this concentration amiodarone HCL was not toxic to dopaminergic neurons. Interestingly, the effect seen in hESC was intermediate between NSCs and dopaminergic neurons. To confirm the specificity of effect of amiodarone treatment on NSCs and rule out the possibility that the different media contributed to the protection seen for dopaminergic neurons, we derived neurons in defined media (Swistowski et al. (2009) PLoS One 4: e6233) and treated them with amiodarone HCL. Like neurons derived by PA6 conditioned medium, neurons generated in defined media were not susceptible to amiodarone toxicity (data not shown).

Effects of Amiodarone HCl on Glia (Non-Neuronal) Cells

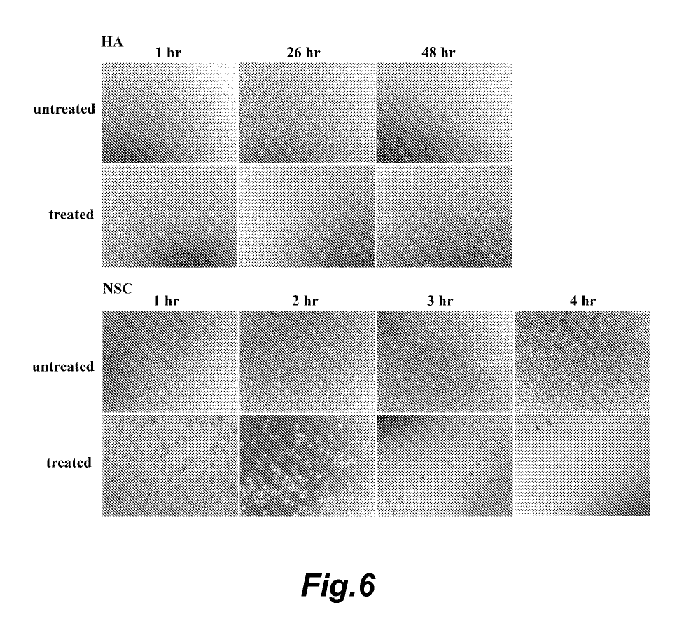

To further confirm the specificity of amiodarone HCl's toxicity on NSCs but not cells differentiated from NSCs, we tested the effect of amiodarone HCl on human fetal-derived astrocytes (Konnikova et al. (2003) BMC Cancer 3: 23), a non-neuronal cell type in the nervous system. As seen in FIG. 6, amiodarone HCl did not cause astrocyte cell death up to 48 hours after treatment, whereas once again massive cell death occurred in similarly treated NSCs within one hour of drug administration. As an additional control we also tested the effect of amiodarone HCl on an immortal cell line 2102Ep cells (Andrews et al. (1982) Int J Cancer 29: 523-531). Like terminally differentiated dopaminergic neurons and astrocytes, no effect was found on 2102Ep cells 48 hours after treatment (data not shown).

Pathways Activated by Amiodarone HCl

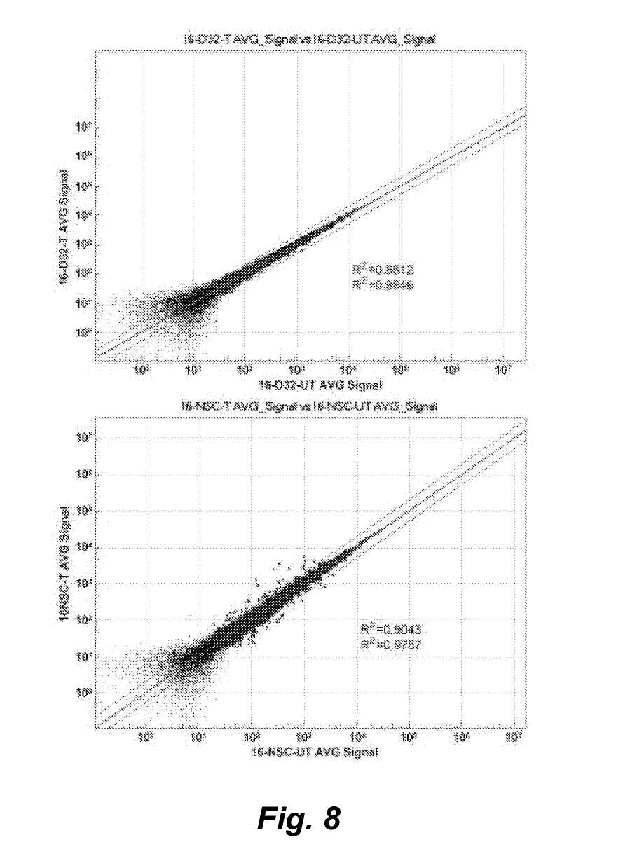

In order to validate that the observed cell death was specific to the action of amiodarone HCL, and possibly dissect the mechanism of action of this compound, we performed a gene expression analysis of NSCs and postmitotic neurons receiving amiodarone HCL. Given that changes in gene expression profiles will likely be seen after a short period exposure to drugs, and that most cells had undergone cell death in as little as 8 hours (FIG. 4, panels E-G), we compared gene expression of cells prior to and after 4 hours of exposure to the drugs. The dataset generated from the expression analysis, along with quality control data and the numbers of genes altered are provided in FIG. 8.

Gene Set Enrichment Analysis (GSEA) was conducted to identify pathways, biological process and molecular functions that are enriched in genes differentially expressed by NSCs or dopaminergic neurons treated with amiodarone HC. In this method, all the genes are ranked according to the differential expression between two classes, and the Kolmogorov-Smirnoff test is used to determine the statistical correlation of the ranked gene list to the gene set of a given biological process, pathway or molecular function. The comparative results are then measured by a non-parametric, running sum statistic termed the enrichment score. The enrichment score significance is assessed by 1,000 permutation tests to compute the enrichment p-value. Table 4 lists the pathways, biological process, and molecular functions that are significantly enriched (P value<0.05) in differentially expressed genes between drug-treated NSCs and non-treated NSCs.

TABLE 4
Pathways enriched in NSC with and without amiodarone treatment

| | | | |
|---|---|---|---|
| Name | Size | Nes | Nom P-Val |

Activities enriched in treated NSCs

| | | | |
|---|---|---|---|
| Transcription Corepressor Activity | 77 | 1.691614 | 0 |
| Serine Hydrolase Activity | 16 | 1.672039 | 0 |
| Transcription Repressor Activity | 124 | 1.652917 | 0 |
| Serine Type Peptidase Activity | 16 | 1.649524 | 0 |
| Cysteine Type Peptidase Activity | 42 | 1.575049 | 0 |
| Endopeptidase Activity | 60 | 1.465786 | 0 |
| Gtpase Regulator Activity | 106 | 1.411686 | 0 |
| Peptidase Activity | 95 | 1.400299 | 0 |
| Enzyme Regulator Activity | 234 | 1.304946 | 0.013514 |
| Cysteine Type Endopeptidase Activity | 31 | 1.621405 | 0.017857 |
| Specific Rna Polymerase Ii | 24 | 1.539421 | 0.018182 |
| Transcription Factor Activity | | | |
| Protein Tyrosine Phosphatase Activity | 35 | 1.461157 | 0.029412 |
| Protease Inhibitor Activity | 20 | 1.558038 | 0.036364 |
| Phosphoprotein Phosphatase Activity | 56 | 1.509656 | 0.037037 |
| Transcription Factor Binding | 251 | 1.247996 | 0.041667 |
| Dna Binding | 439 | 1.231744 | 0.049383 |
| Oxidoreductase Activity Go 0016705 | 17 | 1.545216 | 0.0625 |
| Enzyme Inhibitor Activity | 74 | 1.362072 | 0.063492 |
| Exonuclease Activity | 17 | 1.423085 | 0.065574 |
| Transcription Cofactor Activity | 186 | 1.217252 | 0.08 |
| Deoxyribonuclease Activity | 18 | 1.356687 | 0.087719 |
| Phosphoric Ester Hydrolase Activity | 105 | 1.309918 | 0.089552 |
| Transcription Factor Activity | 251 | 1.217464 | 0.089552 |
| Phosphoric Monoester Hydrolase | 80 | 1.310033 | 0.092105 |
| Activity | | | |
| Substrate Specific Channel Activity | 40 | 1.299635 | 0.107143 |
| Guanyl Nucleotide Exchange Factor | 40 | 1.286659 | 0.109375 |
| Activity | | | |
| Phosphoric Diester Hydrolase Activity | 24 | 1.244372 | 0.140351 |
| Rna Polymerase Ii Transcription | 126 | 1.227469 | 0.140845 |
| Factor Activity | | | |
| Hydrolase Activity Acting On Ester | 178 | 1.18083 | 0.147059 |
| Bonds | | | |
| Protein Complex Binding | 33 | 1.311813 | 0.155172 |
| Gtpase Activator Activity | 48 | 1.185584 | 0.166667 |
| Ion Channel Activity | 39 | 1.254979 | 0.175439 |
| Sh3 Sh2 Adaptor Activity | 27 | 1.235181 | 0.20339 |
| Ion Transmembrane Transporter | 102 | 1.185398 | 0.215385 |
| Activity | | | |
| Molecular Adaptor Activity | 31 | 1.189241 | 0.216667 |
| Secondary Active Transmembrane | 18 | 1.220784 | 0.226415 |
| Transporter Activity | | | |
| Isomerase Activity | 25 | 1.153866 | 0.236364 |
| Anion Transmembrane Transporter | 19 | 1.216016 | 0.241379 |
| Activity | | | |
| Hydrolase Activity Hydrolyzing O | 22 | 1.219036 | 0.245283 |
| Glycosyl Compounds | | | |
| Small Gtpase Regulator Activity | 53 | 1.109262 | 0.295082 |
| Protein Binding Bridging | 37 | 1.103927 | 0.327586 |
| Motor Activity | 21 | 1.104728 | 0.333333 |
| Growth Factor Binding | 19 | 1.135168 | 0.351852 |
| Structural Constituent Of Muscle | 17 | 1.105955 | 0.363636 |
| Substrate Specific Transmembrane | 142 | 1.03954 | 0.380952 |
| Transporter Activity | | | |
| Metal Ion Transmembrane Transporter | 47 | 1.035591 | 0.403226 |
| Activity | | | |
| Structural Constituent Of | 32 | 1.041064 | 0.412698 |
| Cytoskeleton | | | |
| Hydrolase Activity Acting On | 30 | 1.048988 | 0.416667 |
| Glycosyl Bonds | | | |
| Lyase Activity | 42 | 1.018233 | 0.424242 |
| Transition Metal Ion Binding | 72 | 1.036514 | 0.431034 |
| Transmembrane Transporter Activity | 156 | 1.010714 | 0.434783 |
| Oxidoreductase Activity | 180 | 0.986486 | 0.472973 |
| S Adenosylmethionine Dependent | 18 | 0.997775 | 0.473684 |
| Methyltransferase Activity | | | |
| Adenyl Ribonucleotide Binding | 118 | 0.969607 | 0.476923 |
| Cation Transmembrane Transporter | 81 | 1.012556 | 0.478873 |
| Activity | | | |
| Zinc Ion Binding | 55 | 0.979693 | 0.482759 |
| Actin Filament Binding | 19 | 1.029427 | 0.508197 |
| Ras Gtpase Activator Activity | 22 | 0.981198 | 0.510204 |
| Enzyme Binding | 136 | 0.96839 | 0.538462 |
| Nuclease Activity | 43 | 0.939313 | 0.538462 |
| Transmembrane Receptor Protein | 30 | 0.953258 | 0.542373 |
| Tyrosine Kinase Activity | | | |
| Adenyl Nucleotide Binding | 122 | 0.948526 | 0.544118 |
| Mrna Binding | 17 | 1.006026 | 0.546875 |
| Rho Gtpase Activator Activity | 16 | 0.966356 | 0.565217 |
| Methyltransferase Activity | 29 | 0.904404 | 0.596491 |
| Small Gtpase Binding | 29 | 0.917909 | 0.6 |
| Oxidoreductase Activity Go 0016616 | 34 | 0.910647 | 0.61017 |
| Rna Splicing Factor Activity | 17 | 0.954187 | 0.61194 |
| Transesterification Mechanism | | | |
| Gtpase Binding | 30 | 0.958559 | 0.612245 |
| Translation Regulator Activity | 36 | 0.921559 | 0.612903 |
| Single Stranded Dna Binding | 29 | 0.901535 | 0.616667 |
| Cation Channel Activity | 32 | 0.902297 | 0.618182 |
| Oxidoreductase Activity Acting On | 37 | 0.865561 | 0.627119 |
| Ch Oh Group Of Donors | | | |
| Gated Channel Activity | 29 | 0.872611 | 0.672414 |
| Substrate Specific Transporter | 167 | 0.926322 | 0.676056 |
| Activity | | | |
| Nucleotide Binding | 161 | 0.893161 | 0.686567 |
| Hematopoietin Interferon Class D200 | 15 | 0.794707 | 0.694915 |
| Domain Cytokine Receptor Activity | | | |
| Protein Domain Specific Binding | 45 | 0.864267 | 0.7 |
| Purine Nucleotide Binding | 150 | 0.887336 | 0.701493 |
| Purine Ribonucleotide Binding | 146 | 0.903366 | 0.710526 |
| Atp Binding | 111 | 0.881342 | 0.723077 |
| Monovalent Inorganic Cation | 23 | 0.785686 | 0.725807 |
| Transmembrane Transporter Activity | | | |
| Translation Factor Activity Nucleic | 34 | 0.809989 | 0.737705 |
| Acid Binding | | | |
| Transferase Activity Transferring | 16 | 0.843519 | 0.741379 |
| Sulfur Containing Groups | | | |
| Active Transmembrane Transporter | 61 | 0.849457 | 0.742424 |
| Activity | | | |
| Sequence Specific Dna Binding | 40 | 0.824428 | 0.754717 |
| Protein Tyrosine Kinase Activity | 41 | 0.781789 | 0.766667 |
| Protein Kinase Activity | 213 | 0.877212 | 0.774648 |
| Transferase Activity Transferring | 30 | 0.795054 | 0.8 |
| One Carbon Groups | | | |
| General Rna Polymerase Ii | 25 | 0.772008 | 0.807018 |
| Transcription Factor Activity | | | |
| Receptor Signaling Protein Activity | 58 | 0.780308 | 0.808824 |
| Protein Serine Threonine Kinase | 162 | 0.82687 | 0.811594 |
| Activity | | | |
| Ubiquitin Protein Ligase Activity | 38 | 0.753572 | 0.833333 |
| Structure Specific Dna Binding | 46 | 0.761353 | 0.838235 |
| Protein Kinase Binding | 43 | 0.750128 | 0.842857 |
| Transmembrane Receptor Protein | 37 | 0.756431 | 0.846154 |
| Kinase Activity | | | |
| Actin Binding | 55 | 0.745498 | 0.851852 |
| Small Conjugating Protein Ligase | 40 | 0.71703 | 0.852459 |
| Activity | | | |
| Calmodulin Binding | 20 | 0.720394 | 0.859649 |
| Acid Amino Acid Ligase Activity | 45 | 0.777716 | 0.86 |
| Small Protein Conjugating Enzyme | 41 | 0.731379 | 0.867647 |
| Activity | | | |
| Transferase Activity Transferring | 37 | 0.675291 | 0.887097 |
| Groups Other Than Amino Acyl | | | |
| Groups | | | |
| Inorganic Cation Transmembrane | 35 | 0.680157 | 0.894737 |
| Transporter Activity | | | |
| Endonuclease Activity | 21 | 0.528946 | 0.9 |
| Hydro Lyase Activity | 17 | 0.556421 | 0.907407 |
| Transferase Activity Transferring | 22 | 0.660566 | 0.913793 |
| Alkyl Or Aryl Other Than Methyl | | | |
| Groups | | | |
| Protein C Terminus Binding | 58 | 0.667476 | 0.923077 |
| Nuclear Hormone Receptor Binding | 21 | 0.651485 | 0.928571 |
| Transcription Activator Activity | 131 | 0.781159 | 0.942029 |
| Structural Molecule Activity | 153 | 0.74319 | 0.942029 |
| Ligase Activity Forming Carbon | 55 | 0.693735 | 0.948276 |
| Nitrogen Bonds | | | |
| Hormone Receptor Binding | 22 | 0.620983 | 0.949153 |
| Kinase Binding | 49 | 0.630089 | 0.95 |
| Phosphatase Regulator Activity | 20 | 0.455989 | 0.95 |
| Transferase Activity Transferring | 73 | 0.670065 | 0.965517 |
| Glycosyl Groups | | | |
| Identical Protein Binding | 212 | 0.716257 | 0.96875 |
| Translation Initiation Factor Activity | 22 | 0.444906 | 0.981818 |
| Protein Serine Threonine Phosphatase | 18 | 0.494246 | 0.983607 |
| Activity | | | |
| Ribonuclease Activity | 19 | 0.487593 | 0.983607 |
| Transcription Coactivator Activity | 97 | 0.543888 | 1 |
| Ligase Activity | 79 | 0.467311 | 1 |
| Carbon Oxygen Lyase Activity | 21 | 0.404755 | 1 |

Activities Enriched In Untreated Nscs

| | | | |
|---|---|---|---|
| Rna Helicase Activity | 23 | −1.67979 | 0 |
| Atp Dependent Rna Helicase Activity | 17 | −1.62568 | 0 |
| Calcium Ion Binding | 50 | −1.59294 | 0 |
| Phosphotransferase Activity | 16 | −1.66506 | 0.019231 |
| Phosphate Group As Acceptor | | | |
| Rna Dependent Atpase Activity | 18 | −1.8058 | 0.021739 |
| Atp Dependent Helicase Activity | 24 | −1.49938 | 0.027778 |
| Nucleobase Nucleoside Nucleotide | 22 | −1.41684 | 0.047619 |
| Kinase Activity | | | |
| Protein Heterodimerization Activity | 53 | −1.3399 | 0.051282 |
| Cytokine Activity | 32 | −1.38085 | 0.078947 |
| Helicase Activity | 46 | −1.37398 | 0.081081 |
| Transmembrane Receptor Activity | 150 | −1.13198 | 0.108108 |
| Oxidoreductase Activity Acting On | 17 | −1.2992 | 0.131579 |
| The Ch Ch Group Of Donors | | | |
| Phospholipid Binding | 31 | −1.25532 | 0.151515 |
| Growth Factor Activity | 23 | −1.25435 | 0.181818 |
| N Acetyltransferase Activity | 17 | −1.282 | 0.195652 |
| Guanyl Nucleotide Binding | 33 | −1.26525 | 0.196078 |
| Lipid Binding | 53 | −1.14676 | 0.209302 |
| Tubulin Binding | 41 | −1.16862 | 0.219512 |
| Acetyltransferase Activity | 21 | −1.23949 | 0.222222 |
| Ion Binding | 164 | −1.04371 | 0.222222 |
| N Acyltransferase Activity | 19 | −1.20066 | 0.238095 |
| Pyrophosphatase Activity | 178 | −1.05291 | 0.25 |
| Cytokine Binding | 20 | −1.17419 | 0.269231 |
| Damaged Dna Binding | 18 | −1.1681 | 0.270833 |
| G Protein Coupled Receptor Activity | 47 | −1.11789 | 0.289474 |
| Magnesium Ion Binding | 43 | −1.1144 | 0.315789 |
| Phospholipase Activity | 22 | −1.11677 | 0.32 |
| Dna Dependent Atpase Activity | 18 | −1.20048 | 0.326087 |
| Hormone Activity | 17 | −1.19559 | 0.333333 |
| Cation Binding | 122 | −1.03286 | 0.333333 |
| Receptor Binding | 186 | −1.0604 | 0.357143 |
| Phosphoinositide Binding | 16 | −1.12034 | 0.375 |
| Oxidoreductase Activity Acting On | 21 | −1.09204 | 0.378378 |
| Nadh Or Nadph | | | |
| Metallopeptidase Activity | 23 | −1.06001 | 0.378378 |
| Atpase Activity Coupled | 73 | −1.06751 | 0.394737 |
| Hydrogen Ion Transmembrane | 20 | −1.07671 | 0.395349 |
| Transporter Activity | | | |
| Rhodopsin Like Receptor Activity | 23 | −1.04927 | 0.410256 |
| Chromatin Binding | 28 | −1.01492 | 0.413043 |
| Peptide Binding | 35 | −0.93863 | 0.416667 |
| Lipase Activity | 22 | −1.05475 | 0.428571 |
| Receptor Activity | 228 | −1.04375 | 0.428571 |
| Transferase Activity Transferring | 321 | −1.00233 | 0.434783 |
| Phosphorus Containing Groups | | | |
| Carbohydrate Binding | 29 | −0.97286 | 0.452381 |
| Heparin Binding | 18 | −1.01491 | 0.461538 |
| Enzyme Activator Activity | 90 | −1.02383 | 0.470588 |
| Protein Dimerization Activity | 119 | −1.01681 | 0.475 |
| Kinase Activity | 280 | −0.9611 | 0.47619 |
| Kinase Regulator Activity | 31 | −0.92865 | 0.52381 |
| Cofactor Binding | 17 | −0.93262 | 0.545455 |
| Hydrolase Activity Acting On Acid | 180 | −0.99065 | 0.548387 |
| Anhydrides | | | |
| Pattern Binding | 22 | −0.89988 | 0.555556 |
| Glycosaminoglycan Binding | 22 | −0.91273 | 0.560976 |
| Ras Gtpase Binding | 21 | −0.95668 | 0.564103 |
| Amine Transmembrane Transporter | 18 | −0.90019 | 0.575 |
| Activity | | | |
| Udp Glycosyltransferase Activity | 23 | −0.94593 | 0.589744 |
| Polysaccharide Binding | 22 | −0.96752 | 0.604651 |
| Amino Acid Transmembrane | 16 | −0.94549 | 0.613636 |
| Transporter Activity | | | |
| Gtp Binding | 32 | −0.96951 | 0.622222 |
| Protein Kinase Regulator Activity | 27 | −0.88093 | 0.636364 |
| Carboxylic Acid Transmembrane | 20 | −0.83731 | 0.641026 |
| Transporter Activity | | | |
| Exopeptidase Activity | 18 | −0.91499 | 0.642857 |
| Signal Sequence Binding | 15 | −0.82865 | 0.642857 |
| Kinase Inhibitor Activity | 16 | −0.80018 | 0.682927 |
| Phosphotransferase Activity Alcohol | 251 | −0.92896 | 0.7 |
| Group As Acceptor | | | |
| Transferase Activity Transferring | 43 | −0.91463 | 0.717949 |
| Acyl Groups | | | |
| Transferase Activity Transferring | 52 | −0.88142 | 0.727273 |
| Hexosyl Groups | | | |
| Organic Acid Transmembrane | 20 | −0.81932 | 0.763158 |
| Transporter Activity | | | |
| Gtpase Activity | 74 | −0.81096 | 0.763158 |
| Protein Kinase Inhibitor Activity | 16 | −0.78055 | 0.764706 |
| Atpase Activity | 91 | −0.82526 | 0.782609 |
| Receptor Signaling Protein Serine | 26 | −0.79537 | 0.782609 |
| Threonine Kinase Activity | | | |
| Hydrolase Activity Acting On Carbon | 28 | −0.78229 | 0.790698 |
| Nitrogen But Not Peptide Bonds | | | |
| Protein Homodimerization Activity | 75 | −0.80097 | 0.8 |
| Nucleoside Triphosphatase Activity | 166 | −0.89946 | 0.827586 |
| Microtubule Binding | 29 | −0.71432 | 0.864865 |
| Atpase Activity Coupled To | 15 | −0.54303 | 0.880952 |
| Transmembrane Movement Of Ions | | | |
| Unfolded Protein Binding | 37 | −0.65076 | 0.882353 |
| Nucleotidyltransferase Activity | 37 | −0.6782 | 0.921053 |
| Structural Constituent Of Ribosome | 69 | −0.63433 | 0.939394 |
| Cytoskeletal Protein Binding | 117 | −0.78998 | 0.944444 |
| Protein N Terminus Binding | 29 | −0.55418 | 0.944444 |
| Hydrolase Activity Acting On Acid | 25 | −0.40967 | 0.975 |
| Anhydrides Catalyzing | | | |
| Transmembrane Movement Of | | | |
| Substances | | | |
| Primary Active Transmembrane | 26 | −0.38584 | 0.97619 |
| Transporter Activity | | | |
| Dna Helicase Activity | 21 | −0.55417 | 0.977778 |
| Electron Carrier Activity | 57 | −0.63073 | 0.978261 |
| Rna Binding | 206 | −0.66586 | 1 |
| Double Stranded Dna Binding | 28 | −0.4584 | 1 |
| Atpase Activity Coupled To | 26 | −0.37429 | 1 |
| Movement Of Substances | | | |

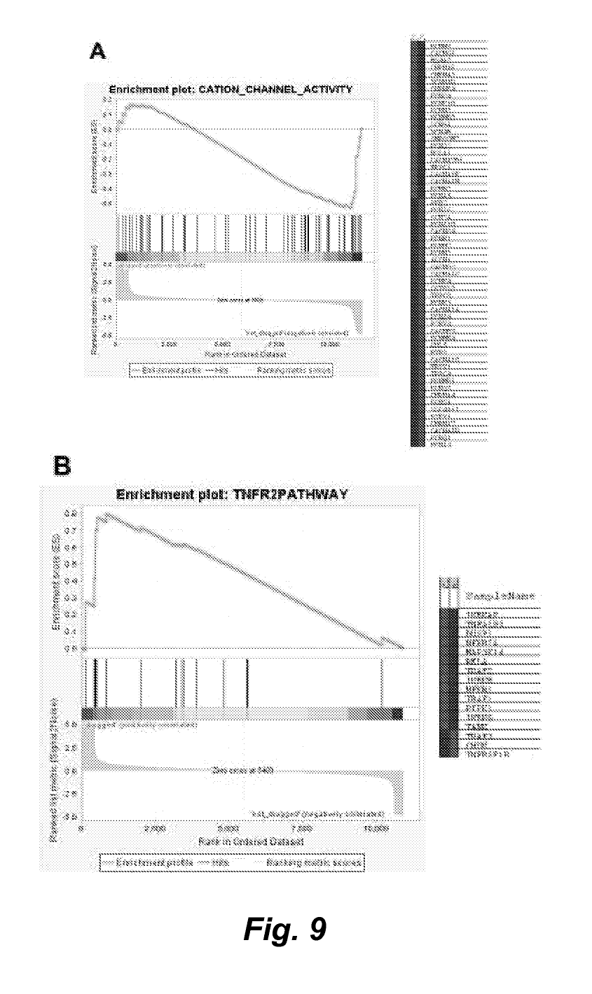

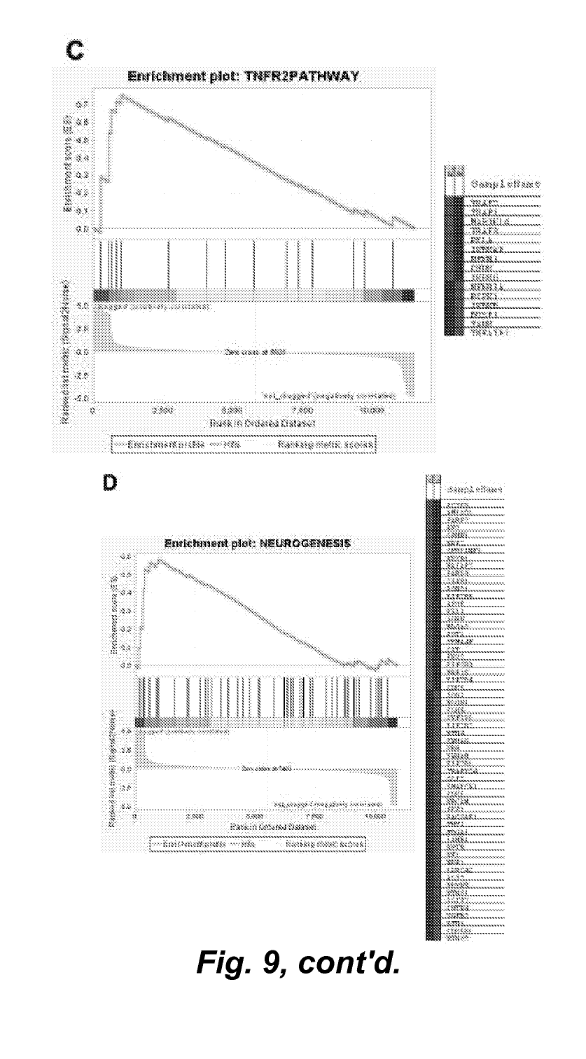

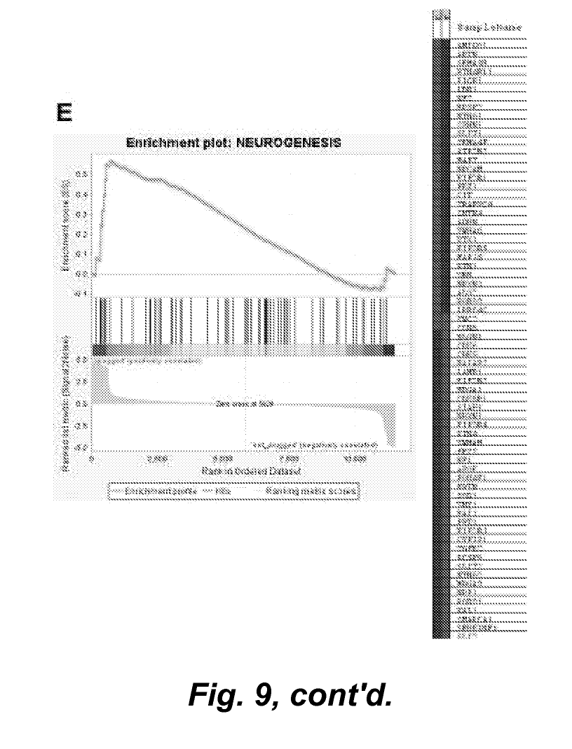

Table 5 lists the pathways, biological process, and molecular functions that are significantly enriched (P value<0.05) in differentially expressed genes between drug-treated dopaminergic neurons and untreated populations. As shown in FIG. 9, GSEA analysis revealed that cation channel activity was higher in both cohorts of untreated NSCs and dopaminergic neurons, while it was low in susceptible NSCs treated with amiodarone HCL (FIG. 9, panel A). We noted that the tumor necrosis factor receptor 2 (TNFR2) pathway and neurogenic pathways were enriched in drug-treated NSCs (P value<0.035, FIG. 9, panels B-E), but the two pathways were not enriched in NSCs and dopaminergic neurons prior to drug treatment. These results in their aggregate suggest that cationic channels, TNFR2-related pathways and neurogenic pathways may have important implications in the response of NSCs to amiodarone HCL drug treatment.

TABLE 5
Activities enriched in DA neurons with and without amiodarone treatment.
Activities Enriched In Treated DA Neurons

| Name | Size | Nes | Nom P-Val |
|---|---|---|---|
| Secondary Active Transmembrane | 21 | 1.632949 | 0 |
| Transporter Activity | | | |
| Protein Serine Threonine Kinase | 164 | 1.386265 | 0.027778 |
| Activity | | | |
| Sequence Specific Dna Binding | 37 | 1.388153 | 0.056338 |
| Phosphotransferase Activity Alcohol | 249 | 1.237356 | 0.059524 |
| Group As Acceptor | | | |
| Anion Transmembrane Transporter | 25 | 1.432292 | 0.065574 |
| Activity | | | |
| Lipase Activity | 21 | 1.421672 | 0.067797 |
| Active Transmembrane Transporter | 71 | 1.375974 | 0.082192 |
| Activity | | | |
| Monovalent Inorganic Cation | 25 | 1.3181 | 0.122807 |
| Transmembrane Transporter Activity | | | |
| Receptor Signaling Protein Serine | 29 | 1.268038 | 0.157895 |
| Threonine Kinase Activity | | | |
| Protein Kinase Activity | 214 | 1.195546 | 0.176471 |
| Peptide Binding | 41 | 1.226343 | 0.181818 |
| Structural Constituent Of Muscle | 21 | 1.208285 | 0.207547 |
| Structure Specific Dna Binding | 44 | 1.183457 | 0.230769 |
| Receptor Signaling Protein Activity | 59 | 1.148881 | 0.236842 |
| Ligase Activity Forming Carbon | 58 | 1.126519 | 0.242857 |
| Nitrogen Bonds | | | |
| Small Conjugating Protein Ligase | 43 | 1.14368 | 0.243243 |
| Activity | | | |
| Phosphoric Diester Hydrolase Activity | 26 | 1.182993 | 0.25 |
| Rhodopsin Like Receptor Activity | 28 | 1.14436 | 0.25 |
| Ligase Activity | 83 | 1.139904 | 0.25 |
| Small Protein Conjugating Enzyme | 44 | 1.146143 | 0.257576 |
| Activity | | | |
| Deoxyribonuclease Activity | 17 | 1.315043 | 0.258621 |
| Acid Amino Acid Ligase Activity | 48 | 1.17974 | 0.28 |
| Ubiquitin Protein Ligase Activity | 41 | 1.138988 | 0.301587 |
| Atp Binding | 114 | 1.076941 | 0.318841 |
| Oxidoreductase Activity Go 0016616 | 34 | 1.058256 | 0.353846 |
| Atpase Activity Coupled To | 29 | 1.065411 | 0.355932 |
| Movement Of Substances | | | |
| Dna Binding | 431 | 1.034326 | 0.359551 |
| Nuclease Activity | 40 | 1.071979 | 0.360656 |
| Phospholipase Activity | 20 | 1.124752 | 0.363636 |
| Udp Glycosyltransferase Activity | 27 | 1.093145 | 0.366667 |
| Hydrogen Ion Transmembrane | 21 | 1.077725 | 0.366667 |
| Transporter Activity | | | |
| Kinase Activity | 277 | 1.036083 | 0.367816 |
| Rna Polymerase Ii Transcription | 124 | 1.041575 | 0.371429 |
| Factor Activity | | | |
| Hydrolase Activity Acting On Acid | 28 | 1.094199 | 0.376812 |
| Anhydrides Catalyzing | | | |
| Transmembrane Movement Of | | | |
| Substances | | | |
| Double Stranded Dna Binding | 26 | 1.071692 | 0.37931 |
| Enzyme Activator Activity | 92 | 1.048951 | 0.382353 |
| Transmembrane Receptor Activity | 170 | 1.054918 | 0.382716 |
| Inorganic Cation Transmembrane | 39 | 1.065105 | 0.40625 |
| Transporter Activity | | | |
| Enzyme Inhibitor Activity | 70 | 1.052384 | 0.409091 |
| Transferase Activity Transferring | 34 | 1.050073 | 0.415385 |
| Groups Other Than Amino Acyl | | | |
| Groups | | | |
| Endonuclease Activity | 20 | 0.985867 | 0.419355 |
| Cytokine Activity | 30 | 1.062567 | 0.421875 |
| Oxidoreductase Activity Acting On | 37 | 1.053572 | 0.424242 |
| Ch Oh Group Of Donors | | | |
| Oxidoreductase Activity Acting On | 18 | 1.027969 | 0.428571 |
| The Ch Ch Group Of Donors | | | |
| Primary Active Transmembrane | 29 | 1.055886 | 0.430556 |
| Transporter Activity | | | |
| Gtpase Activity | 79 | 1.01517 | 0.442857 |
| Transferase Activity Transferring | 39 | 1.002919 | 0.451613 |
| Acyl Groups | | | |
| Damaged Dna Binding | 17 | 1.035788 | 0.45283 |
| Phosphatase Regulator Activity | 21 | 1.041847 | 0.467742 |
| N Acetyltransferase Activity | 15 | 1.019038 | 0.473684 |
| Structural Constituent Of Cytoskeleton | 34 | 0.970783 | 0.482759 |
| Adenyl Ribonucleotide Binding | 120 | 0.984786 | 0.506667 |
| G Protein Coupled Receptor Activity | 55 | 0.985169 | 0.514706 |
| Protease Inhibitor Activity | 20 | 0.98644 | 0.516667 |
| Transmembrane Receptor Protein | 33 | 0.964975 | 0.516667 |
| Kinase Activity | | | |
| N Acyltransferase Activity | 17 | 0.988866 | 0.519231 |
| Enzyme Regulator Activity | 228 | 0.979318 | 0.520548 |
| Hormone Activity | 18 | 0.97489 | 0.523077 |
| General Rna Polymerase Ii | 24 | 0.923838 | 0.530612 |
| Transcription Factor Activity | | | |
| Pyrophosphatase Activity | 183 | 0.953558 | 0.54321 |
| Receptor Activity | 256 | 0.97382 | 0.5625 |
| Purine Ribonucleotide Binding | 149 | 0.957684 | 0.5625 |
| Transferase Activity Transferring | 21 | 0.940302 | 0.566667 |
| Sulfur Containing Groups | | | |
| Magnesium Ion Binding | 43 | 0.940149 | 0.567568 |
| Integrin Binding | 17 | 0.914059 | 0.571429 |
| Substrate Specific Transporter | 211 | 0.92899 | 0.573171 |
| Activity | | | |
| Acetyltransferase Activity | 19 | 0.947398 | 0.6 |
| Single Stranded Dna Binding | 26 | 0.953332 | 0.605634 |
| Protein Homodimerization Activity | 82 | 0.881709 | 0.621212 |
| Protein Domain Specific Binding | 52 | 0.935722 | 0.628571 |
| Lipid Transporter Activity | 15 | 0.919447 | 0.62963 |
| Identical Protein Binding | 217 | 0.952644 | 0.630137 |
| Protein Dimerization Activity | 128 | 0.924809 | 0.636364 |
| Ion Transmembrane Transporter | 139 | 0.927608 | 0.64 |
| Activity | | | |
| Transferase Activity Transferring | 58 | 0.926571 | 0.642857 |
| Hexosyl Groups | | | |
| Nucleotide Binding | 164 | 0.932434 | 0.643836 |
| Transferase Activity Transferring | 317 | 0.94993 | 0.64557 |
| Phosphorus Containing Groups | | | |
| Zinc Ion Binding | 52 | 0.89596 | 0.647059 |
| Translation Regulator Activity | 35 | 0.876409 | 0.661017 |
| Translation Factor Activity Nucleic | 33 | 0.845424 | 0.681159 |
| Acid Binding | | | |
| Carbohydrate Binding | 28 | 0.888154 | 0.688525 |
| Adenyl Nucleotide Binding | 125 | 0.884667 | 0.694118 |
| Microtubule Binding | 30 | 0.812262 | 0.696429 |
| Substrate Specific Transmembrane | 182 | 0.907061 | 0.697368 |
| Transporter Activity | | | |
| Transcription Cofactor Activity | 186 | 0.877451 | 0.7 |
| Transcription Coactivator Activity | 98 | 0.877727 | 0.708333 |
| Sulfotransferase Activity | 17 | 0.88001 | 0.719298 |
| Endopeptidase Activity | 66 | 0.813589 | 0.720588 |
| Metallopeptidase Activity | 21 | 0.873137 | 0.733333 |
| Neurotransmitter Binding | 15 | 0.789043 | 0.754717 |
| Neurotransmitter Receptor Activity | 15 | 0.797205 | 0.757576 |
| Lyase Activity | 47 | 0.864626 | 0.758065 |
| Transmembrane Transporter Activity | 196 | 0.898466 | 0.759494 |
| Transcription Repressor Activity | 122 | 0.855519 | 0.76 |
| Nucleoside Triphosphatase Activity | 173 | 0.873411 | 0.761905 |
| Purine Nucleotide Binding | 154 | 0.861007 | 0.763158 |
| Hydrolase Activity Acting On Acid | 185 | 0.868824 | 0.770115 |
| Anhydrides | | | |
| Motor Activity | 22 | 0.771596 | 0.77193 |
| Calcium Channel Activity | 18 | 0.753383 | 0.781818 |
| Hydro Lyase Activity | 19 | 0.773039 | 0.792453 |
| Calcium Ion Binding | 56 | 0.791332 | 0.805556 |
| Hydrolase Activity Acting On Carbon | 15 | 0.666303 | 0.826923 |
| Nitrogen But Not Peptide Bonds In | | | |
| Linear Amides | | | |
| Transmembrane Receptor Protein | 27 | 0.785587 | 0.828947 |
| Tyrosine Kinase Activity | | | |
| Ion Binding | 166 | 0.812988 | 0.833333 |
| Molecular Adaptor Activity | 35 | 0.768201 | 0.84507 |
| Gtp Binding | 33 | 0.769023 | 0.848485 |
| Guanyl Nucleotide Exchange Factor | 36 | 0.770802 | 0.857143 |
| Activity | | | |
| Carbon Oxygen Lyase Activity | 23 | 0.740198 | 0.86 |
| Transferase Activity Transferring | 79 | 0.829738 | 0.864865 |
| Glycosyl Groups | | | |
| Cation Binding | 125 | 0.782515 | 0.894737 |
| Protein N Terminus Binding | 30 | 0.716727 | 0.910714 |
| Calmodulin Binding | 19 | 0.691734 | 0.910714 |
| Guanyl Nucleotide Binding | 34 | 0.720005 | 0.913044 |
| Hydrolase Activity Acting On Ester | 185 | 0.77466 | 0.924051 |
| Bonds | | | |
| Transcription Corepressor Activity | 76 | 0.695835 | 0.942857 |
| Atpase Activity Coupled To | 17 | 0.593549 | 0.944444 |
| Transmembrane Movement Of Ions | | | |
| Unfolded Protein Binding | 38 | 0.625776 | 0.955224 |
| Structural Molecule Activity | 157 | 0.732434 | 0.963415 |
| Transferase Activity Transferring | 21 | 0.628434 | 0.968254 |
| Alkyl Or Aryl Other Than Methyl | | | |
| Groups | | | |
| Actin Filament Binding | 19 | 0.313769 | 0.983871 |
| Mrna Binding | 18 | 0.408682 | 0.984375 |
| Transcription Factor Binding | 252 | 0.742013 | 0.987013 |
| Phosphoric Ester Hydrolase Activity | 113 | 0.695887 | 0.9875 |
| Atpase Activity | 93 | 0.65281 | 1 |
| Rna Binding | 209 | 0.440631 | 1 |
| Translation Initiation Factor Activity | 22 | 0.331139 | 1 |

Activities enriched in untreated DA neurons

| Name | SIZE | NES | NOM p-val |
|---|---|---|---|
| Gtpase Binding | 29 | −1.61975 | 0 |
| Hydrolase Activity Hydrolyzing O | 25 | −1.47947 | 0 |
| Glycosyl Compounds | | | |
| Ras Gtpase Binding | 21 | −1.58473 | 0.02 |
| Cation Channel Activity | 57 | −1.39352 | 0.027778 |
| Small Gtpase Binding | 28 | −1.4967 | 0.047619 |
| Gated Channel Activity | 54 | −1.49125 | 0.051282 |
| Ion Channel Activity | 66 | −1.19958 | 0.060606 |
| Hydrolase Activity Acting On | 33 | −1.41401 | 0.08 |
| Glycosyl Bonds | | | |
| Nucleobase Nucleoside Nucleotide | 22 | −1.36784 | 0.081081 |
| Kinase Activity | | | |
| Voltage Gated Channel Activity | 31 | −1.28394 | 0.085714 |
| Kinase Binding | 51 | −1.31564 | 0.1 |
| Exopeptidase Activity | 18 | −1.3223 | 0.105263 |
| Potassium Channel Activity | 23 | −1.32529 | 0.111111 |
| Metal Ion Transmembrane Transporter | 76 | −1.26886 | 0.111111 |
| Activity | | | |
| Substrate Specific Channel Activity | 68 | −1.22108 | 0.129032 |
| Chromatin Binding | 28 | −1.23921 | 0.142857 |
| Gtpase Activator Activity | 48 | −1.22185 | 0.157895 |
| Phosphoprotein Phosphatase Activity | 63 | −1.15038 | 0.162162 |
| Electron Carrier Activity | 55 | −1.20425 | 0.166667 |
| Voltage Gated Cation Channel | 29 | −1.17396 | 0.166667 |
| Activity | | | |
| Transition Metal Ion Binding | 69 | −1.09776 | 0.171429 |
| Protein C Terminus Binding | 60 | −1.19544 | 0.212121 |
| Helicase Activity | 46 | −1.18729 | 0.216216 |
| Protein Complex Binding | 35 | −1.13316 | 0.222222 |
| Enzyme Binding | 136 | −1.04 | 0.233333 |
| Ras Gtpase Activator Activity | 23 | −1.14374 | 0.243902 |
| S Adenosylmethionine Dependent | 18 | −1.2358 | 0.25 |
| Methyltransferase Activity | | | |
| Ligand Dependent Nuclear Receptor | 18 | −1.17725 | 0.25641 |
| Activity | | | |
| Protein Kinase Binding | 44 | −1.11558 | 0.258065 |
| Ligand Gated Channel Activity | 17 | −1.23641 | 0.263158 |
| Cation Transmembrane Transporter | 111 | −1.08038 | 0.291667 |
| Activity | | | |
| Rho Gtpase Activator Activity | 15 | −1.15992 | 0.297297 |
| Rna Dependent Atpase Activity | 17 | −1.10955 | 0.318182 |
| Dna Helicase Activity | 22 | −1.12245 | 0.319149 |
| Atp Dependent Helicase Activity | 23 | −1.13329 | 0.324324 |
| Methyltransferase Activity | 29 | −1.089 | 0.340909 |
| Auxiliary Transport Protein Activity | 15 | −1.14107 | 0.347826 |
| Transcription Factor Activity | 246 | −1.02235 | 0.35 |
| Rna Helicase Activity | 22 | −1.05343 | 0.365854 |
| Growth Factor Activity | 25 | −1.07927 | 0.367347 |
| Small Gtpase Regulator Activity | 54 | −1.03771 | 0.387097 |
| Phosphotransferase Activity | 16 | −1.05813 | 0.431818 |
| Phosphate Group As Acceptor | | | |
| Protein Tyrosine Phosphatase Activity | 39 | −1.01857 | 0.432432 |
| Tubulin Binding | 40 | −0.98943 | 0.444444 |
| Peptidase Activity | 101 | −0.9811 | 0.444444 |
| Cytokine Binding | 19 | −0.96435 | 0.459459 |
| Gtpase Regulator Activity | 102 | −0.96851 | 0.461538 |
| Actin Binding | 60 | −1.00722 | 0.464286 |
| Transferase Activity Transferring One | 30 | −0.99648 | 0.466667 |
| Carbon Groups | | | |
| Receptor Binding | 189 | −0.98651 | 0.47619 |
| Protein Serine Threonine Phosphatase | 18 | −0.99073 | 0.48718 |
| Activity | | | |
| Protein Binding Bridging | 40 | −0.9268 | 0.5 |
| Polysaccharide Binding | 19 | −0.92306 | 0.5 |
| Atp Dependent Rna Helicase Activity | 16 | −1.02238 | 0.52381 |
| Sh3 Sh2 Adaptor Activity | 30 | −0.87549 | 0.542857 |
| Pattern Binding | 19 | −0.94385 | 0.567568 |
| Glycosaminoglycan Binding | 19 | −0.90975 | 0.571429 |
| Phosphoric Monoester Hydrolase | 86 | −0.9735 | 0.612903 |
| Activity | | | |
| Hydrolase Activity Acting On Carbon | 29 | −0.85011 | 0.634146 |
| Nitrogen But Not Peptide Bonds | | | |
| Cofactor Binding | 16 | −0.85706 | 0.636364 |
| Protein Tyrosine Kinase Activity | 39 | −0.83815 | 0.657143 |
| Oxidoreductase Activity Go 0016705 | 19 | −0.86064 | 0.675676 |
| Growth Factor Binding | 17 | −0.83417 | 0.705882 |
| Signal Sequence Binding | 15 | −0.81856 | 0.707317 |
| Cysteine Type Peptidase Activity | 43 | −0.84396 | 0.714286 |
| Cytoskeletal Protein Binding | 122 | −0.90039 | 0.730769 |
| Dna Dependent Atpase Activity | 18 | −0.83316 | 0.783784 |
| Oxidoreductase Activity | 186 | −0.91477 | 0.818182 |
| Cysteine Type Endopeptidase Activity | 32 | −0.8093 | 0.818182 |
| Organic Acid Transmembrane | 24 | −0.72342 | 0.833333 |
| Transporter Activity | | | |
| Lipid Binding | 55 | −0.79643 | 0.852941 |
| Ribonuclease Activity | 17 | −0.61729 | 0.875 |
| Protein Kinase Regulator Activity | 25 | −0.73437 | 0.885714 |
| Rna Splicing Factor Activity | 17 | −0.56751 | 0.891892 |
| Transesterification Mechanism | | | |
| Specific Rna Polymerase Ii | 23 | −0.64118 | 0.904762 |
| Transcription Factor Activity | | | |
| Nuclear Hormone Receptor Binding | 20 | −0.49296 | 0.904762 |
| Transcription Activator Activity | 130 | −0.84114 | 0.90625 |
| Isomerase Activity | 28 | −0.60478 | 0.90625 |
| Serine Type Endopeptidase Activity | 18 | −0.65879 | 0.911111 |
| Kinase Regulator Activity | 30 | −0.69893 | 0.911765 |
| Phospholipid Binding | 31 | −0.68686 | 0.914894 |
| Atpase Activity Coupled | 74 | −0.7071 | 0.925926 |
| Carboxylic Acid Transmembrane | 24 | −0.69599 | 0.947368 |
| Transporter Activity | | | |
| Phosphoinositide Binding | 16 | −0.56037 | 0.953488 |
| Serine Hydrolase Activity | 21 | −0.63038 | 0.955556 |
| Amino Acid Transmembrane | 20 | −0.49382 | 0.955556 |
| Transporter Activity | | | |
| Hormone Receptor Binding | 21 | −0.47737 | 0.969697 |
| Protein Heterodimerization Activity | 56 | −0.79087 | 0.971429 |
| Oxidoreductase Activity Acting On | 21 | −0.49945 | 0.975 |
| Nadh Or Nadph | | | |
| Serine Type Peptidase Activity | 21 | −0.65289 | 0.97561 |
| Nucleotidyltransferase Activity | 34 | −0.49704 | 1 |
| Amine Transmembrane Transporter | 22 | −0.41078 | 1 |
| Activity | | | |
| Structural Constituent Of Ribosome | 66 | −0.26757 | 1 |

Based upon the GSEA results, we wanted to test our hypothesis that amiodarone HCL toxicity may act via specific cationic channels. We reasoned that a higher basal expression level of cation channels would render cells more susceptible to the channel blocking effect of amiodarone HCL seen in the GSEA data. Indeed, the role of amiodarone HCL in blocking multiple cation channels has been previously described (Deffois et al. (1996) Neurosci Lett 220: 117-120; Sheldon et al. (1989) Circ Res 65: 477-482; Yeih et al. (2000) Heart 84: E8; Papp et al. (1996) J Cardiovasc Pharmacol Ther 1: 287296; Holmes et al. (2000) J Cardiovasc Electrophysiol 11: 11521158; Das and Sarkar (2003) Pharmacol Res 47: 447461; Calkins et al. (1992) J Am Coll Cardiol 19: 347-352; Xi et al. (1992) J Biol Chem 267: 25025-25031; Sato et al. (1994) J Pharmacol Exp Ther 269: 1213-1219). To interrogate the susceptibility of both NSCs and dopaminergic neurons to amiodarone HCL-induced channel blocking, we examined differences in the expression of ion channels in both NSCs and dopaminergic neurons (Table 6). Comparison of gene expression profiles indicate that both the SLC2A1 and CLICl receptor subunit transcripts are expressed at significantly higher levels in NSCs but not in differentiated neurons, suggesting that NSCs may be more sensitive to the channel-effects of amiodarone HCL. Interestingly, published reports show that hESCs, which are intermediately affected by treatment with amiodarone HCL relative to NSCs and DA neurons (FIG. 5), express SLC2A1 at higher levels than DA neurons, but less than the expression seen in NSCs (expression levels of 317 and 103.2 from two independent lines of BG01, sample 131 and 122, respectively, seen in Liu et al. (2006) BMC Dev Biol 6: 20).

TABLE 6
Ion channel gene expression in NSCs with and without amiodarone
HCl treatment compared to similarly treated dopaminergic neurons.

| | | | | Treated | Untreated |
|---|---|---|---|---|---|
| | | Treated | Untreated | DA | DA |
| Category | Gene | NSC | NSC | Neuron | Neuron |
| H ion | ATP6V1A | 1155.9 | 1093.8 | 2621.5 | 2441.8 |
| transporters | ATP6V1B2 | 385.4 | 331 | 724.8 | 438.2 |
| | ATP6V0D1 | 3355 | 2760.4 | 2920.7 | 2452.5 |
| | ATP5B | 7030.3 | 5745.1 | 5217.1 | 4313.4 |
| | ATP6V0A2 | 190.2 | 163 | 131.3 | 108 |
| | SLC2A11 | 22.8 | 31.1 | .9 | 34.9 |
| | SLC35B1 | 1769.8 | 1380.4 | 1450.4 | 1122.8 |
| | SLC2A1 | 1274.8 | 1574.5 | 87.1 | 86.7 |
| Amine | SLC1A2 | 15.7 | 21.3 | 809.7 | 681 |
| transporters | SLC1A3 | 552.1 | 433.5 | 2926 | 2578.1 |
| | SLC6A3 | 21.2 | 4.8 | 23.4 | 10.5 |
| | SLC6A9 | 329.2 | 251.5 | 36570.5 | 489.3 |
| | SLC6A12 | 9.6 | 7.4 | 10.8 | 10.4 |
| | ATP1A1 | 586.9 | 615.7 | 426.9 | 341.4 |
| | ATP1A1 | 786.4 | 725.1 | 471 | 399.4 |
| Cl channels | CLCN6 | 349.8 | 311.8 | 998.5 | 745.9 |
| | CLCN7 | 1305.5 | 903.2 | 1261 | 1058.4 |
| | CLCN3 | 566.5 | 440.4 | 541.2 | 497.2 |
| | CLCN2 | 41.6 | 30.2 | 24 | 21.7 |
| | CLIC1 | 122.2 | 94.9 | 22.9 | 18 |
| Voltage | SCN9A | 1.9 | 2.6 | 245.7 | 175.1 |
| gated Na | SCN1A | 20.5 | 7.6 | 115.6 | 110.2 |
| channels | SCN3A | 5.7 | 6.3 | 60.4 | 72.3 |
| Amiloride | ACCN1 | 16.5 | 15.4 | 387.4 | 360.1 |
| sensitive Na | ACCN3 | 11 | 5.1 | 35.7 | 38.1 |
| channels | ACCN2 | 185.7 | 167.4 | 862.7 | 837.1 |
| Rectifier K | KCND2 | 2.3 | 8.9 | 269.9 | 225.5 |
| channels | KCNQ2 | 138.1 | 137 | 1753.5 | 1378.2 |
| | KCNC4 | 4.5 | 5.8 | 58.7 | 38.7 |
| | KCNJ4 | 15.9 | 11.6 | 66.1 | 64.3 |
| | KCNQ3 | 3.4 | 5.5 | 23.2 | 25.4 |
| | KCNG1 | 150.4 | 92.7 | 351 | 365 |
| | KCNF1 | 189.2 | 152 | 479.7 | 457.6 |
| | KCNJ11 | 13.5 | 17.7 | 44.4 | 26.5 |
| | KCNJ6 | 317.3 | 271.5 | 297.7 | 256.7 |
| | KCNQ2 | 919.6 | 796.2 | 434.4 | 420.4 |
| Delayed | KCNA5 | 24.2 | 0.4 | 99.9 | 84 |
| rectifier | KCNS1 | 5.2 | 2.2 | 20.3 | 39.9 |
| K channels | KCNH2 | 25.6 | 32 | 165.6 | 151.9 |
| | KCNB1 | 32.8 | 34.2 | 145.4 | 111.5 |
| | KCNB2 | 25 | 28.7 | 94.5 | 79.1 |
| | KCNH2 | 11.2 | 5.8 | 21 | 14.2 |
| Ca activated K | KCNN1 | 6.5 | 0.6 | 48.4 | 28.9 |
| channels | KCNN3 | 0.7 | 6.3 | 181.7 | 127.1 |
| | KCNN2 | 15.8 | 12.3 | 55.6 | 50.1 |
| | KCNMB1 | 62.9 | 60.4 | 44.8 | 72.6 |
| Calcium | CACNB2 | 7.8 | 12 | 155.2 | 156.9 |
| channels | CACNG2 | 2.5 | 11.5 | 123.8 | 115.6 |
| | CACNA1A | 2 | 10.1 | 64.4 | 62.6 |
| | CACNA1C | 25.9 | 29.3 | 146.9 | 138.8 |
| | CACNA1H | 122.7 | 87.8 | 268.9 | 220.4 |

The TNFR2 pathway, also identified in the GSEA analysis as being selectively enriched in NSCs treated with amiodarone HCL (FIG. 9, panels B-C), has been shown to trigger cellular apoptosis (Tartaglia et al. (1993) J Biol Chem 268: 18542-18548). To elucidate the downstream activators of cell death in the amiodarone HCL-treated samples, we sought to examine transcription factors that were either activated or repressed four hours after exposure to the drug. To be more specific, we searched for transcription factors that were changed in NSCs after exposure to amiodarone HCl but showed no change in differentiated cells after treatment with equivalent amounts of the drug. Table 7 lists the transcription factors. As can be seen in Table 7, amiodarone HCL treatment in NSCs significantly up regulated Fos, FosB, and DDIT3, transcription factors known to participate in TNFα receptor-mediated apoptosis through formation of the DNA-binding complex AP-1 (Zhang et al. (2009) Int J Cancer 124: 1980-1989; Dong et al. (2006) J Cell Biochem 98: 1495-1506; Baumann et al. (2003) Oncogene 22: 1333-1339; Fujii et al. (2008) Infect Immun 76: 3679-3689). Notably, genes thought to induce and promote apoptosis through the intrinsic mitochondrial apoptotic pathway, such as KLF 10 (Jin et al. (2007) FEBS Lett 581: 3826-3832), were not altered in differentiated cells or in treated versus untreated cells. Since amiodarone HCL is known to exert its cytotoxic effect through the extrinsic, caspase-9 independent apoptotic pathway (Yano et al. (2008) Apoptosis 13: 543-552) our microarray results confirm that the differential cytotoxic effect seen in NSCs treated with amiodarone HCL is due to specific activation of extrinsic apoptosis pathways resulting from exposure to the drug.

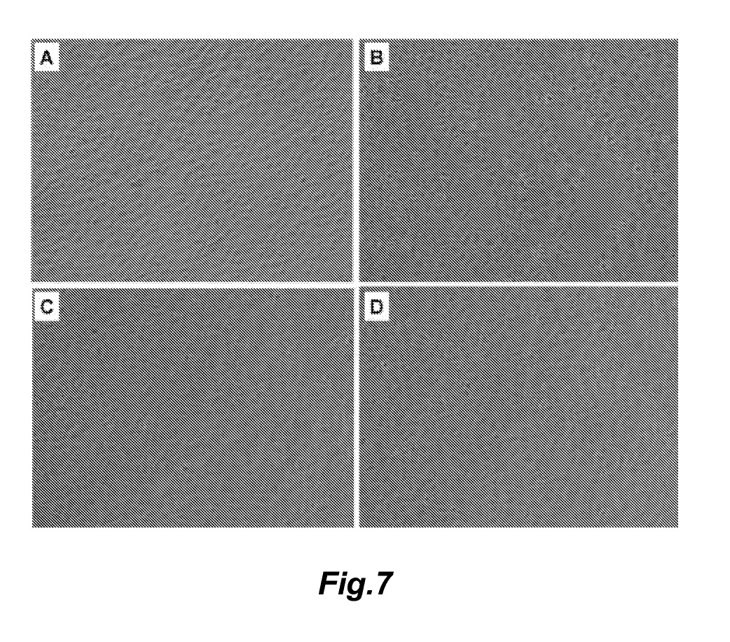

Our microarray data showed a number of genes in the TNFα pathway were highly expressed in amiodarone HCl-treated NSCs. We therefore examined whether cell death in NSCs upon amiodarone HCl exposure could be due to the activation of soluble TNFα signaling pathways. Three dosages of soluble TNFα (0.1 mM, 1 mM and 10 mM) were tested in NSC culture for 48 hours. Under these conditions we did not observe differences in cell death between treated and untreated cells (FIG. 7).

TABLE 7
Transcription factors that are differentially expressed in
NSCs with and without amiodarone HCl treatment.

| | | Treated | Untreated | Treated | Untreated |
|---|---|---|---|---|---|
| Category | Gene | NSC | NSC | Neuron | Neuron |
| Gene expression | EGR1 | 3100 | 339 | 109 | 128 |
| higher in treated | DDIT3 | 2270 | 269 | 170 | 145 |
| NSCs | FOS | 1160 | 123 | 1980 | 1410 |
| | FOSB | 174 | 1 | 346 | 185 |
| | TAF5L | 77.4 | 1 | 17.5 | 17.6 |
| | RELB | 53.6 | 1 | 31.9 | 27.6 |
| | IRF1 | 38.9 | 1 | 1 | 1 |
| | KLF10 | 38.1 | 1 | 1 | 1 |
| | MEF2C | 33.1 | 1 | 49.8 | 48.2 |
| | ZNF197 | 32.8 | 1 | 5.1 | 1 |
| | THRB | 32.7 | 1 | 1 | 23.1 |
| | TEF | 32.4 | 1 | 23.5 | 1 |
| | CREBL1 | 32.2 | 1 | 1 | 1 |
| | HIRA | 31.5 | 1 | 24.2 | 27.4 |
| | MYEF2 | 30.6 | 1 | 25.3 | 1 |
| | NR4A2 | 30.4 | 1 | 212 | 183 |
| | L3MBTL | 27.5 | 1 | 97.7 | 99.9 |
| Gene expression | DLX1 | 1550 | 1650 | 1 | 1 |
| higher in NSC | ETS1 | 952 | 891 | 1 | 1 |
| compared to | HOXA2 | 770 | 810 | 1 | 1 |
| neuron | ETV4 | 672 | 688 | 1 | 1 |
| | TEAD4 | 235 | 213 | 1 | 1 |
| | HOXB4 | 198 | 251 | 1 | 1 |
| | HOXB3 | 186 | 214 | 1 | 1 |
| | MEOX1 | 178 | 180 | 1 | 1 |
| | EGR2 | 155 | 44.1 | 1 | 1 |
| | FOXL2 | 120 | 115 | 1 | 1 |
| | TGIF1 | 117 | 62.8 | 1 | 1 |
| | ELF4 | 103 | 116 | 1 | 1 |
| | PAX8 | 98.9 | 104 | 1 | 1 |
| | PRDM1 | 94.7 | 94.5 | 1 | 1 |
| | ELK3 | 89.9 | 90.9 | 1 | 1 |
| | TEAD3 | 87 | 75.1 | 1 | 1 |
| | E2F8 | 82.7 | 73.8 | 1 | 1 |
| | SALL1 | 80.9 | 103 | 1 | 17 |
| | FOXD1 | 79.7 | 99.7 | 1 | 1 |
| | PBX2 | 70.8 | 52.2 | 1 | 11.3 |
| | HOXD3 | 70.2 | 64.1 | 1 | 1 |
| | PRRX2 | 67.5 | 80.8 | 1 | 22.7 |
| | STAT6 | 55.8 | 58 | 1 | 17.1 |
| | DLX2 | 54.3 | 34.1 | 1 | 1 |
| | AFF1 | 50.8 | 58.1 | 1 | 24.9 |
| | HOXB5 | 49.9 | 69.2 | 1 | 1 |
| | EN1 | 48.7 | 59.3 | 1 | 1 |
| | ZSCAN29 | 47.6 | 42.6 | 1 | 1 |
| | TBX2 | 46.9 | 29.3 | 1 | 1 |
| | GATA2 | 46.2 | 45.8 | 1 | 1 |
| | HOXB2 | 1100 | 981 | 24 | 25 |
| | NR1D2 | 44.9 | 30.7 | 1 | 1 |
| | NRK | 43.8 | 42.3 | 1 | 1 |
| | GLI2 | 227 | 239 | 5.2 | 27.5 |
| | RUNX1 | 43.3 | 48.6 | 1 | 1 |
| | LHX8 | 42.9 | 28.5 | 1 | 1 |
| | MSX2 | 41.9 | 39.4 | 1 | 1 |
| | HCLS1 | 40 | 23.1 | 1 | 1 |
| | ELK4 | 39.3 | 26 | 1 | 1 |
| | FOXF2 | 39.3 | 29.7 | 1 | 1 |
| | IRF1 | 38.9 | 1 | 1 | 1 |
| | KLF10 | 38.1 | 1 | 1 | 1 |
| | HEY2 | 37.9 | 28.3 | 1 | 1 |
| | ZNF274 | 37.8 | 31.1 | 1 | 1 |
| | FOXA2 | 36.8 | 27.2 | 1 | 1 |
| | ASCL2 | 36 | 32.9 | 1 | 1 |
| | TAF13 | 35.2 | 35.8 | 1 | 1 |
| | ETV1 | 34.4 | 39.5 | 1 | 1 |
| | HOXA13 | 33.2 | 35.9 | 1 | 1 |
| | ERG | 32.9 | 24.9 | 1 | 17.5 |
| | NFATC4 | 32.8 | 34.5 | 1 | 1 |
| | THRB | 32.7 | 1 | 1 | 23.1 |
| | NFATC3 | 32.4 | 44.7 | 1 | 1 |
| | SIX4 | 185 | 193 | 5.7 | 1 |
| | CREBL1 | 32.2 | 1 | 1 | 1 |
| | ZNF367 | 30.9 | 25.9 | 1 | 1 |
| | ELF5 | 30.8 | 30.2 | 1 | 1 |
| | HOXB1 | 30.6 | 23.7 | 1 | 1 |
| | EGR1 | 3100 | 339 | 109 | 128 |
| | ZNF85 | 93.8 | 57.2 | 5.6 | 32.3 |
| | E2F7 | 711 | 556 | 43 | 65.5 |
| | DDIT3 | 2270 | 269 | 170 | 145 |
| | HMGB2 | 2170 | 2000 | 163 | 163 |
| | NFKB2 | 68 | 62.6 | 5.3 | 24.6 |
| | NR2C2 | 65.3 | 60.9 | 5.7 | 23.2 |
| | TP53 | 267 | 279 | 23.7 | 26.3 |
| | ARID3A | 2200 | 2480 | 201 | 208 |
| | FLI1 | 123 | 147 | 11.3 | 26.8 |
| | FOXM1 | 287 | 271 | 27.7 | 32.2 |
| | E2F2 | 635 | 545 | 63.2 | 60.9 |
| | FOXC1 | 327 | 393 | 32.6 | 25.7 |

### DISCUSSION

Our screening approach provides a new platform technology for using hESCs and purified populations of their differentiated neural derivatives to rapidly screen and identify compounds that exert specific effects on these cell types. This screening approach relies on the observable phenotype of cell death coupled with gene expression analysis to identify pathways of cell-type specific drug activity. To extend its utility, this approach can also provide clues to the molecular mechanisms that participate in stage-specific cytotoxic effects of candidate drugs. We had reasoned that because of fundamental differences in cell cycle and growth factor dependence, there would likely be drugs that were specific to one cell type versus another. Indeed, as expected in our primary screen we identified nine such compounds. Of these initial 9 candidates, 6 compounds demonstrated dose responsive toxicity exclusively in NSC populations. Interestingly, the compounds amiodarone HCL and selamectin had the most dramatic ameliorating effect on NSC survival (FIG. 2). It was surprising to us that none of these compounds were in the expected classes of anti cancer or anti-proliferative agents but instead included anti-parasitic and antiarrhythmic drugs.

We chose to further investigate one of these drugs, amiodarone HCl, which specifically killed NSCs but not dopaminergic neurons differentiated from NSCs. Amiodarone has for decades achieved clinical status as an effective class III antiarrhythmic drug in cardiac patients (Patterson et al. (1983) Circulation 68: 857-864; Flaker et al. (1985) Am Heart J 110: 371-376). Importantly, because it is already approved for clinical use, amiodarone HCL may have clinical applications in cell replacement therapies by selectively removing only the unwanted undifferentiated NSCs during the pre-transplant period.

In order to confirm that the cytotoxic effect seen in the amiodarone HCL-treated NSCs was specific to the activity of the drug, we first sought to determine which cellular pathways were affected in the amiodarone HCL susceptible NSC population relative to unaffected dopaminergic neurons receiving the same treatment (FIG. 9). The GSEA data revealed amiodarone HCL treated samples had significantly reduced expression of factors involved in ion channel activity. Amiodarone is known to specifically block ion channels, which suggests that the effect seen in the drug treated samples is specific to amiodarone HCL activity. To further test this, we reasoned that populations of cells with a greater basal expression of ion channel activity mediators would be most susceptible to drug treatment. Indeed, microarray data confirmed that amiodarone HCL-susceptible NSCs have significantly increased base-line expression of certain ion channels (Table 6, SLC2A1 and CLC1A). It is tantalizing to speculate that amiodarone HCl might also be toxic to other stem cell populations that demonstrate increased ion channel expression relative to their differentiated derivatives, including mesenchymal stem cells (MSCs) and endothelial precursor cells (Wang et al. (2008) Clin Exp Pharmacol Physiol 35: 1077-1084), thus expanding the utility of the automated screening assay described here.

Amiodarone has been shown to exert its cytotoxic effect via a TNF-related signaling pathway that includes caspase-8 mediated apoptosis (Yano et al. (2008) Apoptosis 13: 543-552). Thus, we next wanted to determine whether our assay could detect subtle changes in TNF activity in samples treated with amiodarone HCL. Notably, downstream members of the TNFR2 pathway were significantly augmented in the amiodarone HCL-treated NSC population (FIG. 9). TNFR2 belongs to a class of membrane glycoprotein receptors that specifically bind TNFα. TNFR1 is expressed on most cell types, while TNFR2 expression is restricted to endothelial, hematopoietic and some neuronal populations (McCoy and Tansey (2008) J Neuroinflammation 5: 45; Grell (1995) J Inflamm 47: 8-17). TNFα is a potent pro-inflammatory cytokine with two biologically active forms that are either soluble (solTNF) or membrane bound (tmTNF), and TNFR2 is preferentially activated by tmTNF (Grell et al. (1995) Cell 83: 793-802). It was initially thought that TNFα-mediated signaling downstream of TNFR1 results in apoptosis, while those downstream of TNFR2 induce proliferation (Tartaglia et al. (1991) Proc. Natl. Acad. Sci., USA, 88: 9292-9296). Additional work, however, revealed that in collaboration with TNFR1, TNFα can act upon TNFR2 through a ligand passing mechanism and trigger apoptosis (Id.).

These published reports in their aggregate support that TNFR2 can lower the threshold of bioavailable TNFα needed to cause apoptosis through TNFR1 thus amplifying extrinsic cell death pathways. In fact, short term treatment of patients with amiodarone leads to a significant decrease in the patient's serum TNFα concentrations while paradoxically the amiodarone toxicity is exerted through TNF-mediated apoptotic pathways (Hirasawa et al. (2009) Circ J73: 639646). These observations are explained by the fact that amiodarone HCL up regulates TNFR2, and TNFR2 is more dependent on ligation with tmTNF than solTNF. To test this model, we treated amiodarone HCL-susceptible NSCs with solTNF.

If amiodarone HCL toxicity is mediated through TNFR2, and TNFR2 is not sensitive to solTNF, then addition of solTNFα should not be cytotoxic to the NSCs. Indeed, three doses of solTNFα (0.1 mM, 1 mM and 10 mM) were tested in NSC culture for 48 hours and no increase in cell death relative to untreated cultures was observed (FIG. 7). This supports published reports that the addition of solTNFα to NSC cultures actually induces proliferation and differentiation (Widera et al. (2006) BMC Neurosci 7: 64; Johansson et al. (2008) Stem Cells 26: 2444-2454; Yin et al. (2008) Stem Cells Dev 17: 5365). Since TNFα is such a potent inducer of apoptosis through TNFR1 death domain signaling, and amiodarone treatment results in the down regulation of TNFα with concomitant upregulation in TNFR2 signaling in NSC alone, it is possible that amiodarone selectively kills NSCs by lowering the threshold of TNFα required to trigger apoptosis in NSCs via upregulation of TNFR2 pathways in NSCs and not dopaminergic neurons.

Our results support our primary goal of identifying a previously approved drug that may allow us to deplete mitotic NSCs from an otherwise differentiated population of dopaminergic neurons, thus ensuring their safety for use in transplantation. Importantly, this automated screening assay allowed us to interrogate some of the specific molecular mechanisms that may be responsible for the targeted cytotoxic effect amiodarone HCL had on NSCs and not cells differentiated from NSCs. While we do not purport to know the molecular mechanisms by which amiodarone HCL leads to the toxicity we observed in NSCs, it is notable that the results of our automated screening, including GSEA and microarray analysis, are all consistent with published literature that implicates the roles of ion channels and TNFα signaling in amiodaronemediated cytotoxicity. This suggests that our automatic screening assay is specifically measuring the effect amiodarone HCL has on different populations of cells. Our methodology can also be easily expanded to other screens in the neural system. For example, we note that purified populations of motor neurons and oligodendrocytes are now readily available from hESCs and our screening strategy can be extended to these cell populations as well.

In conclusion, we describe a method using hESCs and their differentiated neural derivatives that permits the rapid screening of clinically approved drugs for compounds that can be safely used to selectively deplete progenitor cells from a differentiated cell product. Importantly, this approach is adaptable for use in a Chemistry, Manufacture and Control drug screening protocol and may have applications in identifying lineage specific reagents, thus providing additional evidence for the utility of stem cells in screening and discovery paradigms.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of culturing pluripotent stem cells in a feeder-free format compatible with high throughput screening, said method comprising:
providing human embryonic stem cells in a matrigel coated dish; and
culturing said stem cells in medium comprising Dulbecco's Modified Eagle's medium/Ham's F12 supplemented with one or more of the following:
knockout serum replacement;
non-essential amino acids;
L-glutamine;
β-mercaptoethanol;
an antibiotic; and
basic fibroblast growth factor;
wherein said medium is conditioned with embryonic fibroblasts.

2. The method of claim 1, wherein said pluripotent cell is an embryonic stem cell (ESC) or an induced pluripotent stem cell (iPSC).

3-5. (canceled)

6. The method of claim 1, wherein said medium is conditioned with embryonic fibroblasts.

7. The method of claim 1, wherein said knockout serum replacement comprises from about 5% to about 20% of said culture medium.

8. The method, wherein said knockout serum replacement comprises about 20% of said culture medium.

9. The method of claim 1, wherein said non-essential amino acids range from about 1 mM to about 2 mM in said culture medium.

10. (canceled)

11. The method of claim 1, wherein said L-glutamine ranges from about 1 mM to about 8 mM in said culture medium.

12. (canceled)

13. The method of claim 1, wherein said β-mercaptoethanol ranges from about 0.1 mM to about 1 mM in said culture medium.

14. (canceled)

15. The method of claim 1, wherein said antibiotic is Penn-Strep and ranges from about 50 μg/mL to about 100 μg/mL in said culture medium.

16. (canceled)

17. The method of claim 1, wherein said basic fibroblast growth factor ranges from about 4 ng/mL to about 20 ng/mL in said culture medium.

18. (canceled)

19. The method of claim 1, wherein said Dulbecco's Modified Eagle's medium/Ham's F12 medium is supplemented with:
about 20% knockout serum replacement;
about 2 mM non-essential amino acids;
about 4 mM L-glutamine;
about 0.01 mM β-mercaptoethanol;
about 50 μg/mL Penn-Strep; and
about 4 ng/mL basic fibroblast growth factor.

20. A method of culturing neural stem cells (NSCs) in a feeder-free format compatible with high throughput screening, said method comprising:
providing neural stem cells in a fibronectin coated dish; and
culturing said stem cells in medium comprising DMEF/12 supplemented with:
N2 medium;
non-essential amino acids;
bFGF; and
EGF.

21. The method of claim 20, wherein said medium is supplemented with N2 ranging from about 0.5× to about 1×.

22. (canceled)

23. The method of claim 20, wherein said non-essential amino acids range from about 1 mM to about 2 mM in said culture medium.

24. (canceled)

25. The method of claim 20, wherein said bFGF ranges from about 10 ng/mL to about 50 ng/mL in said culture medium.

26. (canceled)

27. The method of claim 20, wherein said EGF ranges from about 10 ng/mL to about 20 ng/mL in said culture medium.

28. (canceled)

29. The method of claim 20, wherein said medium is supplemented with:
about 1×N2 medium;
about 2 mM non-essential amino acids;
about 20 ng/mL of bFGF; and
about 2 ng/mL of EGF.

30. A method of screening an agent for the ability to selectively inhibit the growth and/or proliferation of pluripotent stem cells and/or neural stem cells, said method comprising:
contacting said pluripotent stem cells with said test agent;
contacting a multipotent and/or a terminally differentiated cell with said test agent;
determining the cytotoxicity of said test agent on said pluripotent cell and on said multipotent and/or terminally differentiated cell; and
selecting agents that are preferentially cytotoxic or protective to pluripotent cells over multipotent cells and/or selecting agents that are preferentially cytotoxic or protective to pluripotent cells and/or multipotent cells over terminally differentiated cells.

31. The method of claim 30, wherein said pluripotent cell is an embryonic stem cell (ESC) or an induced pluripotent stem cell (iPSC).

32-34. (canceled)

35. The method of claim 30, wherein multipotent cell is a progenitor cell or a neural stem cell.

36. (canceled)

37. The method of claim 30, wherein said selecting comprises recording the identity of agents that are preferentially cytotoxic to ESCs over NSCs and/or preferentially cytotoxic to ESC and/or NSCs over terminally differentiated cells in a database of agents that to selectively inhibit the growth and/or proliferation of human embryonic stem cells and/or neural stem cells.

38. The method of claim 30, wherein said selecting comprises storing to a computer readable medium, or listing to a computer monitor or to a printout, the identity of agents that are preferentially cytotoxic or protective to ESCs over NSCs and/or preferentially cytotoxic or protective to ESC and/or NSCs over terminally differentiated cells in a database of agents that selectively inhibit the growth and/or proliferation of human embryonic stem cells and/or neural stem cells.

39-40. (canceled)

41. The method of claim 30, wherein said selecting comprises further screening the selected agents for cytotoxic activity on cell lines.

42. The method of claim 30, wherein said method comprises contacting a neural stem cell (NSC) with said test agent, and/or contacting a terminally differentiated cell with said test agent.

43-44. (canceled)

45. The method of claim 30, wherein said determining the cytotoxicity comprises performing one or more assays selected from the group consisting of an ATP assay, a lactate dehydrogenase (LDH) assay, an adenylate kinase (AK) assay, a glucose 6-phosphate dehydrogenase (G6PD) assay, MTT assay, and a MTS assay.

46. The method of claim 30, wherein said selecting comprises identifying the agent as an NSC killer if it shows cytotoxicity against NSCs with at least 1.5 fold or greater potency for NSCs than ESCs or iPSCs and shows at least a 25% reduction in viability of NSCs as compared to a control.

47. (canceled)

48. The method of claim 30, wherein said selecting comprises identifying the agent as an NSC killer if it reduces ATP concentrations with at least 2-fold or more potency for NSCs than ESCs, and that NSC values are 50% or more below a control mean.

49. The method of claim 30, wherein said selecting comprises identifying the agent as an ESC killer if there is any significant selectivity for affecting ATP levels in ESCs over NSCs.

50. The method of claim 30, wherein said contacting an embryonic stem cell comprises culturing said embryonic stem cell according to the method comprising:
providing human embryonic stem cells in a matrigel coated dish; and
culturing said stem cells in medium comprising Dulbecco's Modified Eagle's medium/Ham's F12 supplemented with one or more of the following:
knockout serum replacement;
non-essential amino acids;
L-glutamine;
β-mercaptoethanol;
an antibiotic; and
basic fibroblast growth factor;
wherein said medium is conditioned with embryonic fibroblasts.

51. The method of claim 30, wherein said contacting a neural stem cell comprises culturing said neural stem cell in a method comprising
providing neural stem cells in a fibronectin coated dish; and
culturing said stem cells in medium comprising DMEF/12 supplemented with:
N2 medium;
non-essential amino acids;
bFGF; and
EGF.

52. A method of generating a substantially homogenous population of embryonic stem cells (ESCs), said method comprising:
providing a population of embryonic stem cells and contacting said population with an agent that preferentially kills neural stem cells (NSCs), where said agent is provided in an amount to preferentially kill NSCs without substantially diminishing the population of embryonic stem cells.

53-54. (canceled)

55. A method of generating a substantially homogenous population of adult stem cells derived from human embryonic stem cells (hESCs) or induced pluripotent stem cells, said method comprising:
differentiating adult stem cells from a population of human embryonic stem cells or induced pluripotent stem cells to form a population of adult stem cells; and
contacting said population with an agent that preferentially inhibits the growth or proliferation of human embryonic stem cells or induced pluripotent stem cells remaining in said population, thereby producing a substantially homogenous population of adult stem cells.

56-60. (canceled)

61. A method of generating a substantially homogenous differentiated population of cells derived from human embryonic stem cells (hESCs) or induced pluripotent stem cells, said method comprising:
differentiating cells from a population of human embryonic stem cells or induced pluripotent stem cells to form a population of differentiated cells; and
contacting said population with one or more agents that preferentially inhibit the growth or proliferation of human embryonic stem cells and/or induced pluripotent stem cells, and/or adult stem cells in said population, thereby producing a substantially homogenous differentiated population of cells.

62-69. (canceled)
